(19) United States
(12) Patent Application Publication
(10) Pub. No.: US 2015/0237838 A1
(43) Pub. Date: Aug. 27, 2015

(54) REGULATION OF INSECT POPULATIONS

(71) Applicant: CALIFORNIA INSTITUTE OF TECHNOLOGY, Pasadena, CA (US)

(72) Inventors: BRUCE A. HAY, Encino, CA (US); OMAR S. AKBARI, Pasedena, CA (US)

(21) Appl. No.: 14/631,171

(22) Filed: Feb. 25, 2015

Related U.S. Application Data
(60) Provisional application No. 61/944,847, filed on Feb. 26, 2014.

(51) Int. Cl. A01K 67/033

(52) U.S. Cl. CPC A01K 67/0339; A01K 2227/706

(57) ABSTRACT

The present technology generally relates to compositions, methods, and populations that allow for the regulation of insect population.

### PRIORITY

This application claims the benefit of U.S. provisional application 61/944,847 filed on Feb. 26, 2014, which is hereby incorporated by reference in its entirety.

### STATEMENT REGARDING FEDERALLY SPONSORED R&D

This invention was made with government support under OD003878 awarded by the National Institutes of Health. The government has certain rights in the invention.

### BACKGROUND

1. Field of the Invention

The present technology generally relates to compositions, methods, and populations that allow for the regulation of insect populations.

2. Description of the Related Art

Establishing effective control measures for wild insect populations is a complex and difficult problem. Several different approaches have been used to control insect populations. These approaches utilize different concepts, for example, making genetically modified crops that produce insect specific toxins, and using non-specific pesticides and natural predators.

Yet another approach called the sterile insect technique (SIT) involves releasing an overwhelming number of sterile male insects into the wild resulting in control of wild insect populations as a result of non-productive matings with wild females. This approach involves separation of males from females prior to their release. SIT is a species specific, environmentally benign method of insect population control, whereby overwhelming numbers of sterile insects are released into the wild. Mating of released sterilized males with native females results in the reduction of the females reproductive potential. If enough sterile males are released over a sufficient period of time, the target population can be dramatically suppressed or even eliminated. This powerful approach has proven to be highly successful, and includes area-wide-SIT programs such as the remarkably successful eradication of the screwworm fly Cochliomyia hominivorax Coquerel from the United States, Mexico and Central America (Bushland et al., 1955; Krafsur et al., 1986; Krafsur et al., 1987). There have also been many other successful uses of SIT to control species of fruit flies, including the Mexican fruit fly (Anastrepha ludens) and the Medfly (Ceratitis capitata) (Hendrichs et al., 2002). Additionally, there is currently a long-term sterile insect release program involving the pink bollworm (Pectinophora gossypiella) in the San Joaquin Valley of California. Together, these items demonstrate that SIT can be an effective method for insect population control.

Various types of sex-separation techniques have been developed which include mechanical separation based on natural physical differences between the two sexes. An alternative approach for efficient sex separation that has been effectively used for Anopheles mosquitoes is to link a conditionally lethal allele to the Y chromosome through irradiation-induced chromosomal rearrangements (Curtis et al., 1976).

Another sex separation approach relies on generating transgenic insects that express sex-linked fluorescent markers. This approach has been implemented in the Mediterranean fruit fly by generating transgenic strains harboring selectable markers linked to the Y chromosome (Condon et al., 2007).

A recently developed sex separation technique is known as Release of Insects carrying a Dominant Lethal (RIDL) (Thomas et al., 2000). In this system a gene cassette that confers dominant, drug-repressible, lethality is introduced into the insects. This system involves the expression of a tetracycline repressible transactivator fusion protein (tTa), which binds to the tetracycline-responsive element (tRe), driving expression of a toxin in the absence of the tetracycline. The system is silenced in the presence of tetracycline, and since the toxin is not produced, the progenies survive (characterized herein as a negative selection event, system, or process). In order to mass rear insects in this manner, one need only supplement the diet with tetracycline.

### SUMMARY

In some embodiments, a method of making a genetically engineered insect population is provided. The method comprises transforming a starting insect population with a transformation vector to create a genetically engineered insect population, expanding the genetically engineered insect population in the absence of any selection, and applying a sex-specific selection event to select for a male fraction of the genetically engineered insect population.

In some embodiments, the sex-specific selection event comprises short-term selection. In some embodiments, the sex-specific selection event does not comprise long-term selection. In some embodiments, the sex-specific selection event comprises a positive selection. In some embodiments, the sex-specific selection event does not comprise a negative selection. The negative selection comprises a selection event that must be maintained in order for survival, and stopping administration of a compound to the population results in death within the population. In some embodiments, the method further comprises applying radiation to the male fraction, thereby generating a genetically engineered sterile male insect population. In some embodiments, the starting insect population is a species of one or more of the genus Drosophila, Anopheles, Pectinophora, Anastrepha, or Bombyx.

In some embodiments, a method of using a first insect population for control of a second insect population is provided. The method comprises providing a first insect population. The first insect population comprises a gene encoding a protective protein. The gene comprises a non-native intron within the gene, and the first insect population comprises a genetically engineered sterile male insect population. The method further comprises applying the first insect population to a desired area. The desired area contains a second insect population, thereby controlling, suppressing or eliminating the second insect population in the desired area.

In some embodiments, the desired area is at least one of a household, a park, a residential area, or an urban area. In some embodiments, the method is repeated to achieve a desired level of control of the second insect population. In some embodiments, the desired level of control is a reduction in size of the second insect population by at least about 50%.

In some embodiments, the second insect population comprises an insect that transmits a disease of a mammal. In some embodiments, the second insect population comprises an insect that damages crops.

In some embodiments, a transformation vector is provided. The vector comprises a targeting sequence that allows for insertion of the transformation vector in a genome wherein the genome comprises an insect genome, an antibiotic resistance gene, a sex-specific intron positioned within the antibiotic resistance gene. The intron is excisable by splicing in a sex-specific manner. The excision of the sex-specific intron allows the antibiotic gene to be expressed in a sex-specific manner. The transformation vector further comprises a regulatory element.

In some embodiments, the expression of the antibiotic resistance gene confers resistance to short-term positive sex-specific selection. In some embodiments, the targeting sequence is an inverted terminal repeat. In some embodiments, the targeting sequence is a piggyBac inverted terminal repeat. In some embodiments, the vector further comprises a gene that encodes an enzyme that allows the insertion of the transformation vector in the genome. In some embodiments, the enzyme is a recombinase. In some embodiments, the enzyme is a transposase. In some embodiments, the antibiotic resistance gene confers resistance to neomycin. In some embodiments, the antibiotic resistance gene confers resistance to puromycin. In some embodiments, the intron is from the tra gene. In some embodiments, the intron is from the dsx gene. In some embodiments, the antibiotic resistance gene comprises a tra intron and confers resistance to neomycin upon sex-specific splicing in females but not in males. In some embodiments, the antibiotic resistance gene comprises a tra intron and confers resistance to puromycin upon sex-specific splicing in females but not in males. In some embodiments, the antibiotic resistance gene comprises a dsx intron and confers resistance to neomycin upon sex-specific splicing in males but not in females. In some embodiments, the antibiotic resistance gene comprises a tra intron and confers resistance to puromycin upon sex-specific splicing in males but not in females. In some embodiments, the transformation vector further comprises a transformation marker to identify a genetically engineered insect. In some embodiments, the regulatory element comprises a promoter and a terminator.

In some embodiments, a genetically engineered insect comprising a sex-specific selection element is provided. The sex-specific selection element comprises an antibiotic resistance gene comprising a sex-specific intron. The intron is excisable by splicing in a sex-specific manner. Excision of the sex-specific intron allows the antibiotic resistance gene to be expressed in a sex-specific manner. Expression of the antibiotic resistance gene confers resistance to a positive sex-specific selection.

In some embodiments, the excision of the sex-specific intron occurs only in males. In some embodiments, the antibiotic resistance gene is expressed only in males and confers resistance to neomycin. In some embodiments, the antibiotic resistance gene is expressed only in males and confers resistance to puromycin. In some embodiments, excision of the sex-specific intron occurs only in females. In some embodiments, the antibiotic resistance gene is expressed only in females and confers resistance to neomycin. In some embodiments, the antibiotic resistance gene is expressed only in females and confers resistance to puromycin.

In some embodiments, a male fraction of a genetically engineered insect population comprising a transformation vector is provided. The transformation vector comprises a gene that encodes a protective protein. A sex-specific intron is located within the gene encoding the protective protein. The functionality of the encoded protective protein is dependent upon excision of the sex-specific intron. Excision of the sex-specific intron is determined by the male sex determination machinery.

In some embodiments, male sex determination machinery allows proper removal of the sex-specific intron such that the protective protein confers survival on the male fraction in the presence of a selection event. The selection event is a molecule that is toxic to the male fraction in the absence of the protective protein. In some embodiments, the protective protein is encoded by an antibiotic resistance gene.

In some embodiments, a genetically engineered insect is provided. The insect comprises a normative gene that encodes for a protective protein. The normative gene comprises an intron that is not native to the gene encoding the protective protein. The intron is excisable by splicing in a sex-specific manner. Excision of the intron allows the protective protein to be expressed in a sex-specific manner. Expression of the protective protein confers resistance to a selection event.

### BRIEF DESCRIPTION OF THE DRAWINGS

FIG. 1A is a schematic of a transformation vector for making a genetically engineered insect population.

FIG. 1B is schematic of the method for making a genetically engineered sterile male insect population of some embodiments provided herein.

FIG. 2 is a graph showing selection of wild type versus genetically engineered male insects with puromycin.

FIG. 3 is a graph showing selection of wild type versus genetically engineered female insects with G418.

### DETAILED DESCRIPTION

It has been appreciated that there is a need for a portable, scalable, synthetic, dominant, drug-inducible, sex separation system for multicellular organisms (for example, insects) that can be used for regulating their populations. While the methods noted above were generally effective, there is a constant appearance of insects that evolve resistance to these approaches, resulting in the need for new technologies. Additionally, blanketing crops with insecticides is not cheap or environmentally friendly, and genetically modifying every single crop species is not socially accepted, cannot solve all pest related problems, and remains to be implemented for most crops. Furthermore, while the release of natural predators can be effective, for most insects, no potent natural predator can be easily mass reared and released.

Furthermore, tetracycline has numerous unwanted side effects on insects, such as loss of gut microbiome, loss of symbiotic bacteria, compromised mitochondrial function, and large fitness cost (Zeh et al., Sci. Rep. 2:375 (2012)). Thus, populations that have gone through the negative selection process noted above, can be significantly weaker than expected, meaning that they are not as competitive when released in to a second population to be controlled. Therefore, in some embodiments, what is provided herein is a technology that is easily portable across genera, incorporates a universal, scalable, sex-separation technology that does not rely on the use of cumbersome sex separation devices or approaches, does not cause a large fitness cost to the organism or the environment, and offers a way to effectively control, suppress or eliminate harmful organisms (e.g, insects that transmit diseases of humans) in the wild.

In addition, provided herein are methods for genetically engineering insects in a manner such that survival of the genetically engineered insect is dependent on expression of protein that confers survival capabilities to the insect. In some embodiments, the desired insect is either male or female (depending upon the situation). In some embodiments, this can be achieved by the use of a gene that confers resistance and/or survival to the resulting individual. In some embodiments, this allows one to generate a genetically engineered insect population that can then be used to control, suppress or eliminate one or more insect populations in the wild.

In some embodiments, the organisms provided herein can be used in SIT systems and/or methods. For many of the above-mentioned SIT programs, both sterilized males and females were released to achieve wild population suppression. Although releasing both sexes can work, and is acceptable for SIT in some insects, releasing both sexes generally hampers the effort. For example, releasing sterilized, mixed-sex, Medflies for SIT can be three to five times less effective compared to exclusively releasing sterilized males for wild population suppression (Rendon et al., 2004). This reduction in effectiveness, by the mixed-sex releases, is thought to result from sterile females distracting the sterile males from seeking their wild female intended targets. In addition to distracting the sterile males, females are generally the disease spreading or crop-damaging gender, making exclusive releases of males absolutely necessary in many contexts. Therefore, a prerequisite for SIT in several insects requires that one be able to efficiently separate the sexes prior to release. While other options have been discussed above, various embodiments provided herein allow for a portable, scalable, synthetic, dominant, drug-inducible sex-separation system for insects.

In some embodiments, genetically engineered insect populations can be manipulated to ensure that a primarily single sex population (for example, primarily male) of the insect is selected. This predominantly male population can then be sterilized and used in a desired environment to control other insect populations in the environment.

The present disclosure provides a series of definitions for context of some of the terms and then provides a set of embodiments for various applications of the discoveries. It then provides a set of additional variables that can be applied within the various applications and concludes with a set of Examples.

### DEFINITIONS

The section headings used herein are for organizational purposes only and are not to be construed as limiting the described subject matter in any way. All literature and similar materials cited in this application, including but not limited to, patents, patent applications, articles, books, treatises, and internet web pages are expressly incorporated by reference in their entirety for any purpose. When definitions of terms in incorporated references appear to differ from the definitions provided in the present teachings, the definition provided in the present teachings shall control. It will be appreciated that there is an implied “about” prior to the temperatures, concentrations, times, etc discussed in the present teachings, such that slight and insubstantial deviations are within the scope of the present teachings herein. In this application, the use of the singular includes the plural unless specifically stated otherwise. Also, the use of “comprise”, “comprises”, “comprising”, “contain”, “contains”, “containing”, “include”, “includes”, and “including” are not intended to be limiting. It is to be understood that both the foregoing general description and the following detailed description are exemplary and explanatory only and are not restrictive. Unless defined otherwise, technical and scientific terms used herein have the same meaning as commonly understood by one of ordinary skill in the art to which this invention belongs. See, for example Singleton et al., Dictionary of Microbiology and Molecular Biology 2nd ed., J. Wiley & Sons (New York, N.Y. 1994); Sambrook et al., Molecular Cloning, A Laboratory Manual, Cold Springs Harbor Press (Cold Springs Harbor, N.Y. 1989). For purposes of the present invention, the following terms are defined below. It is to be understood that both the foregoing general description and the following detailed description are exemplary and explanatory only and are not restrictive of the invention as claimed. In this application, the use of the singular includes the plural unless specifically stated otherwise. In this application, the use of “or” means “and/or” unless stated otherwise. Furthermore, the use of the term “including”, as well as other forms, such as “includes” and “included”, is not limiting. Also, terms such as “element” or “component” encompass both elements and components comprising one unit and elements and components that comprise more than one subunit unless specifically stated otherwise. Also, the use of the term “portion” can include part of a moiety or the entire moiety.

As used herein, “positive selection” refers to selection based on protection of individuals with specific genotypes based on their expression of a molecule that protects them from an otherwise lethal challenge mediated by a compound (for example an organic compound) or alteration of the physical environment, such as heat or cold. In short, positive selection refers to situations in which a transgene confers condition-dependent protection from death. In some embodiments, the molecule can be a selection molecule (for example, an organic compound that is used in a selection event) or a selection event generally (such as survival to a change in the environment, such as heat or cold).

As used herein, “negative selection” is selection based on sensitivity of individuals of a particular genotype to the presence or absence of a compound that protects them when present and causes death when absent, or visa versa. In short, negative selection refers to a situation in which the presence confers condition-dependent mortality or disruption in some other essential activity, such as flight, feeding, reproduction. In some embodiments, the toxin is at least one of a cell death protein, a restriction endonuclease, or a microRNA.

In some embodiments, positive selection (for example, resistance to a toxin in the environment) is conferred based on the presence of a functional protective protein within the host. In some embodiments, the protective protein can convert a toxic form of the positive selection molecule into a non-toxic form, thereby allowing survival of an organism with a functional protective protein. In some embodiments the protective protein is a protein that allows for resistance to antibiotics and can be encoded by an antibiotic resistance gene. In some embodiments, the positive selection molecule is neomycin. In some embodiments, the positive selection molecule is puromycin. In some embodiments, the positive selection molecule can be any known in the art.

As used herein, “long-term selection” is defined as selection that is applied over a duration of time in order to be effective. For example, long-term selection can be applied for the entire duration of the developmental life cycle of an organism (for example, an insect), and can be used in the context of negative selection.

As used herein, “short-term selection” refers to applying a selection event just to allow for the selection event itself to occur. In some embodiments, this is normally done for a small part of a developmental stage of the life cycle of an organism (for example, one to all instar stages, or fraction thereof, of development of an insect. In some embodiments, this is done for all larval instar stages. In some embodiments, this can be done by feeding adults to selectively kill off one sex. In some embodiments, short-term selection can be used in the context of positive selection events. Short-term selection need only be applied during the actual selection process.

The term “regulatory element” is used herein to refer to nucleic acid elements that can influence the expression of a coding sequence (for example, a gene) in a particular host organism. These terms are used broadly and cover all elements that promote or regulate transcription, including promoters, core elements required for basic interaction of RNA polymerase and transcription factors, upstream elements, enhancers, and response elements (see, for example, Lewin, “Genes V” (Oxford University Press, Oxford) pages 847-873).

As used herein, the term “sex-specific selection” refers to a system or method arranged such that health or survival of an organism is dependent upon the sex of the organism when the organism is exposed to a selective pressure.

As used herein, “short-term positive sex-specific selection” refers to sex-specific selection using positive selection over a short-term. Short-term denotes that it is adequately long to allow the desired degree of selection, but that it need not be maintained after that period of time in order for the organism to survive.

As used herein, the term “suppress” refers to a reduction. In some embodiments, suppression is complete, although it need not be complete suppression in all embodiments.

As used herein, the term “desired level of control” refers to a reduction in a population size of a multicellular organism (for example, an insect) to a desired amount. For example, the reduction can be at least 10%, 20%, 30%, 40%, 50%, 60%, 70%, 80%, 90, 95%, 98%, 99%, or complete. In some embodiments, the population is a wild population.

As used herein, the term transformation (transform/transforming/transformed) refers to the introduction of a transformation vector (for example, a plasmid) into a host or its progenies. For example, the transformation vector can be introduced into an insect or its progenies by injecting the transformation vector into the germline of the insect or its progenies. In some embodiments, this can be achieved through one of several different methods, such as: using transposon-based vectors, using vectors that integrate site-specifically at a docking site introduced into the strain previously; or vectors that bring about integration following the introduction of a targeted DNA double-stranded break.

As used herein, the term “transformation vector” or “vector” refers to a polynucleotide construct, typically a plasmid, used to transmit genetic material to a host cell. Vectors can also be, for example, viruses, cosmids, or phage. A vector as used herein can be composed of either DNA or RNA. In some embodiments, a vector is composed of DNA. Vectors are preferably capable of autonomous replication in a prokaryote such as E. coli, used for growth. Once integrated into the genome of the eukaryotic organism of interest the vector can be immobile, and incapable of autonomous replication or movement. In some embodiments, a vector contains a targeting sequence. In some embodiments, the vector further comprises an antibiotic resistance gene with a sex-specific intron within the antibiotic resistance gene. The intron is excised by splicing in a sex-specific manner such that the excision of the sex-specific intron allows the antibiotic gene to be expressed in a sex-specific manner. For example, the excision of the sex-specific intron allows for the antibiotic resistance gene to be expressed either in male or female but not both. The vector further comprises regulatory elements for regulating gene expression. In some embodiments, these regulatory elements may drive expression in both sexes equally, or drive expression in a sex-biased or sex-specific manner.

As used herein, the term “targeting sequence” refers a nucleic acid sequence that allows for insertion of the vector into a genome of a multicellular organism (for example, an insect genome).

As used herein, the term “sterile male” refers to the male sex of an organism (for example, an insect) that is unable to generate any progeny of the organism with the female.

As used herein, the term “antibiotic resistance gene” refers to a gene that encodes for a protein that increases the chance of survival to an organism expressing the protein when the organism is exposed to an antibiotic. In some embodiments, the antibiotic gene encodes for a protective protein, which can be a protein that provides protection to the organism from an antibiotic.

As used herein, the term “sex-specific intron” refers to an intron that is excisable out of the coding sequence in a sex-specific manner in an organism (for example, an insect). In some embodiments, the excision is determined by the insect's sex determination machinery. In some embodiments, the sex-specific intron is located within a gene encoding a protective protein. In some embodiments, the protective protein is encoded by an antibiotic resistance gene, and the functionality of the protective protein is dependent upon excision of the sex-specific intron. In some embodiments, the “sex-specific intron” is one that is non-native to the host organism as well as to the gene encoding the protective protein.

ITR denotes an inverted terminal repeat.

As used herein, the term “sex-specific manner” refers to occurrence of a biological process that occurs in one sex of an organism but not in both, for example, the splicing of a sex-specific intron occurs in the male, but not the female of the organism.

A “protective protein” is a protein, which when functional, allows for the organism to thrive and/or survive under a selection event, through the proper functioning of the protective protein. Thus, when the protein is functional, it will confer some survival benefit to the host (in regard to the application of some adverse selection event). When the protein is incomplete or inactive, it will not confer the survival benefit to the host (in regard to the application of the adverse selection event). The protective protein confers some benefit or survival advantage to the organism against the selection molecule.

In regard to “sex determination machinery,” the phrase refers to a group of both sex specifically and sex non-specifically activated genes that are responsible for the regulation of alternative RNA splicing of genes important for sex determination.

Selection event, “selective pressure” or other similar term denotes an environmental variable, which when changed, alters the survival rate or likelihood of an individual or individuals within a population. In some embodiments, selection event can be chemical or biological in nature (and thus the “selection molecule” can be the specific chemical or biological molecule). In some embodiments, the event can be purely environmental, such as oxygen levels, light, fluorescence, temperature, water, etc. Generally, the application of a selection event (for example, via the application of a selection molecule) allows for the selection of the desired organism(s). This can be achieved “negatively” or “positively”.

The term “selection event” denotes the presence of the selection pressure. Thus, a selection event can be the application of a selection molecule (for example chemical agent) to the population, such as the application of an antibiotic to an insect population.

The term “sex-specific selection event” denotes the presence of a selection event in a scenario where the survival rate of the organisms being selected depends upon the sex of the organism. In some embodiments, this dependence is created by the fact that only one sex of the organism will adequately produce a protective protein (or altered protein level) that can help protect them from the selection event.

In some embodiments, a transformation vector can be employed in order to produce the selected population and/or individual and/or execute the various methods provided herein. In some embodiments, the transformation vector can include an antibiotic resistance gene (or, more generally, a gene encoding for a protective protein) and a sex-specific intron positioned within the antibiotic resistance gene (or, more generally, a gene encoding for a protective protein). The intron is configured to be excisable by splicing (by the host) in a sex-specific manner. The transformation vector is configured such that excision of the sex-specific intron allows the antibiotic gene (or, more generally, a gene encoding for a protective protein) to be expressed in a sex-specific manner. Such expression denotes a functional expression of the antibiotic gene (or, more generally, a gene encoding for a protective protein), thereby producing a protein (for example, a protective protein) that confers resistance to the host organism (which can be antibiotic resistance).

In some embodiments, a vector can be employed in order to produce the selected population and/or individual and/or execute the various methods. In some embodiments, the transformation vector can include a gene encoding a protective protein and a sex-specific intron positioned within the protective protein gene. The intron is configured to be excisable by splicing (by the host) in a sex-specific manner. The transformation vector is configured such that excision of the sex-specific intron allows the protective protein to be expressed in a sex-specific manner. Such expression denotes a functional expression of the protective protein, thereby producing a protein that confers a survival benefit to the host organism, in response to a selection event.

In some embodiments, the transformation vector can further comprise a targeting sequence that allows for insertion of the transformation vector into a genome of the host organism. Some embodiments that can be employed include the piggybac transposable element, mariner type transposable elements, and the P-element. Also, plasmids can be site specifically integrated into the genome using attb/attp or even by using CrispR and homologous recombination.

In some embodiments, the transformation vector can further comprise a regulatory element. Exemplary regulatory elements in prokaryotes include promoters, operators and ribosome binding sites. Regulatory elements that are used in eukaryotic cells can include, without limitation, transcriptional and translational control sequences, such as promoters, terminators, enhancers, insulators, splicing signals, polyadenylation signals, terminators, protein degradation signals, internal ribosome-entry element (IRES), 2A sequences, and the like, that provide for and/or regulate expression of a coding sequence and/or production of an encoded polypeptide in a host cell. For example, a promoter is a nucleotide sequence that permits binding of RNA polymerase and directs the transcription of a gene. Typically, a promoter is located in the 5′ non-coding region of a gene, proximal to the transcriptional start site of the gene. Sequence elements within promoters that function in the initiation of transcription are often characterized by consensus nucleotide sequences. Examples of promoters include, but are not limited to, promoters from bacteria, yeast, plants, viruses, and mammals (including humans). A promoter can be inducible, repressible, and/or constitutive. Inducible promoters initiate increased levels of transcription from DNA under their control in response to some change in culture conditions (for example, a change in temperature).

In some embodiments, the antibiotic resistance gene (and/or protective protein) confers resistance to short-term positive sex-specific selection. In some embodiments, this short-term is such that the drug can be exposed during any of the larval stages or potentially even feeding the adult insects.

In some embodiments, the antibiotic resistance (and/or protective protein) gene confers resistance to neomycin. In some embodiments, the antibiotic resistance (and/or protective protein) gene confers resistance to puromycin. Thus, in some embodiments, neomycin and/or puromycin can be used as selection molecules. In some embodiments, other options include one or more of 1) Mutant FabI gene (mFabI) from E. coli genome, which confers triclosan resistance to the host, 2) High expression Dihydrofolate Reductase (DHFR) confers resistance to Methotrexate, 3) High expression of blasticidin resistance gene from Bacillus cereus (bsr), which codes for blasticidin-S deaminase, confers resitance to blasticidin, 4) High expression of the Sh ble gene, first isolated from Streptoalloteichus hindustanus, confers resitance to Zeocin, and 5) High expression of the hygromycin resistance gene confers resistance to Hygromycin B.

In some embodiments, a sex-specific intron is provided within the antibiotic resistance (and/or protective protein) gene that is excised by splicing and allows for the expression of the antibiotic resistance (and/or protective protein) gene in a sex specific manner. In some embodiments, the intron is from the tra gene that is specifically excised by splicing in females. The tra gene encodes a key protein necessary for the correct sexual differentiation of somatic cells in females in some insects. In some embodiments, the intron is from the dsx gene that is specifically excised by splicing in males. The dsx gene encodes a key protein necessary for the correct sexual differentiation of somatic cells in males in some insects. In some embodiments, the gene in question is one of, or corresponds to one or more of, the genes provided in Table 0.1. Table 0.1 provides a list of genes that were bioinformatically predicted to utilize sex specific alternative splicing to regulate sex-specific expression of some of their exons.

In some embodiments, the antibiotic resistance (and/or protective protein) gene comprises a tra intron and confers resistance to neomycin upon sex-specific splicing in females but not in males. In some embodiments, the antibiotic resistance (and/or protective protein) gene comprises a tra intron and confers resistance to puromycin upon sex-specific splicing in females but not in males. In some embodiments, the antibiotic resistance (and/or protective protein) gene comprises a dsx intron and confers resistance to neomycin upon sex-specific splicing in males but not in females. In some embodiments, the antibiotic resistance (and/or protective protein) gene comprises a dsx intron and confers resistance to puromycin upon sex-specific splicing in males but not in females.

In some embodiments, the vector comprises one or more genetic elements that allow the integration of the vector into the genome of a multicellular organism, for example, an insect. In some embodiments, the genetic elements allow for integration into the genome of the insect in either a random or a predetermined manner. For example, the vector can comprise a piggyBac transposable element. Without being limited to any particular theory, it is believed that a vector with the piggyBac transposable element can randomly integrate into the genome of an organism (for example, an insect chromosome). In some embodiments, other examples include the mariner type transposable elements, and the P-element.

In some embodiments, the transformation vector comprises a gene that encodes a recombinase (for example, transposases) that catalyzes the insertion of the vector comprising piggyBac transposable elements into the genome. In some embodiments, the integration site is predetermined, for example, when using a site-specific recombination system such as FLP/FRT or CRE/LOX recombination system to insert the vector into the chromosome of an organism through homologous recombination. In some embodiments transformation vectors can be site specifically integrated into the genome using, for example, attb/attp or even by using CrispR/homologous recombination.

In some embodiments, a method of making a genetically engineered insect population is provided. In some embodiments, the method comprises transforming a starting insect population with a transformation vector to create a transformed insect population. In some embodiments, any of the vectors provided herein can be employed. In some embodiments, the transformed insect population is expanded in the absence of any selection event (for example, in the absence of a negative selection event). A sex-specific selection can be applied following expansion of the transformed insect population.

In some embodiments, selection is a short-term positive sex-specific selection to select for a male fraction of the transformed insect population. This can be achieved by the addition of a selection molecule to the population in some embodiments.

In some embodiments, the selected male fraction is irradiated, thereby generating a genetically engineered sterile male insect population. In some embodiments, the insects are chemically sterilized.

In some embodiments, the vector comprises a transformation marker, for example, a fluorescent protein marker such as dsRed or GFP that can be expressed under the control of suitable regulatory elements. Fluorescent protein can be visualized by illuminating with a suitable excitatory wavelength (for example blue) and observing the fluorescence. Such a marker would allow easy identification of transformants. Other suitable markers for transformation are known in the art, and can be chosen by one of skilled in the art according to need.

In some embodiments, short-term positive sex-specific selection specifically selects for male insects. In some embodiments, short-term positive sex-specific selection specifically selects for female insects.

In some embodiments positive selection is based on functional expression of a gene that encodes for a protective protein (for example, an antibiotic resistance gene). The phrase “functional expression” denotes the concept that when the intron is retained, any resulting protein is nonfunctional (or has a relatively low level of function) in regard to its ability to confer survival to the host organism in the presence of a selection event. In some embodiments, the protective protein (or fragment thereof) is completely nonfunctional. In some embodiments, the protein's protective capabilities are simply reduced so as to still confer some survival benefit to those organisms expressing the protein over other organisms (not expressing the protein). Thus, selection need not always be 100% male or female, but in some embodiments, can simply be and effective biasing. However, in some embodiments, selection will be 100% (or at least above 80%). As described herein, the two different populations can be male or female populations. In some embodiments, the function of the protein is reduced at least 1%, for example, 1, 5, 10, 20, 30, 40, 50, 60, 70, 80, 90, 95, 98, 99, or 100% reduction, including any range between any two of the preceding values and any range above any one of the preceding values.

In some embodiments, the gene that encodes the protective protein has a sex-specific intron inserted within its coding sequence. The intron is excisable out of the gene by sex-specific splicing machinery within the host. In some embodiments, the intron is inserted into the gene of the protective protein; thus, the intron can be “artificial” or nonnative to the protective protein.

Sex-specific splicing machinery includes a group of both sex specifically and sex non-specifically activated genes that are responsible for the regulation of alternative RNA splicing of genes important for sex determination.

The sex specific expression and binding of certain splicing repressors, leads to sex specific retention and/or excision of introns in mRNAs that consequently results in the proteins translated from alternatively spliced mRNAs having differences in their amino acid sequence and, consequently, in their biological functions.

For list of other sex specific intron options see Table 0.1 below:

TABLE 0.1

| GeneName | Coordinates |
|---|---|
| CG32834; CG32833; mir-4939 | chr2R: 18871796-18872837; chr2R: 18870128-18871798; |
| | chr2R: 18872734-18872842 |
| CR43960; CR32657 | chrX: 11938177-12009871; chrX: 11981244-11981728 |
| CR43960; CR32657 | chrX: 11938177-12009871; chrX: 11981244-11981728 |
| CG30438 | chr2R: 1384164-1437048 |
| CR43960; CR32657 | chrX: 11938177-12009871; chrX: 11981244-11981728 |
| CR43960; CR32657 | chrX: 11938177-12009871; chrX: 11981244-11981728 |
| CG13380; CG4174 | chr3L: 18593250-18611043 |
| CG32548 | chrX: 18329748-18334614 |
| CG31709; gcm | chr2L: 9579449-9581742; chr2L: 9578536-9579638 |
| CG33223 | chrX: 8359723-8377918 |
| CG14053 | chrX: 2085984-2087586 |
| qua | chr2L: 17486806-17497203 |
| CG3788 | chr2R: 18819558-18821600 |
| CG13380; CG4174 | chr3L: 18593250-18611043 |
| exu | chr2R: 16554924-16558379 |
| CR42859; CG31686 | chr2L: 2245230-2246207; chr2L: 2244637-2245352 |
| swaPsi | chrX: 6255529-6257343 |
| CG13358; CG13359 | chrX: 697457-701337; chrX: 695702-697503 |
| Ptp61F | chr3L: 1342493-1475249 |
| Ir | chr3R: 18999595-19011364 |
| orb | chr3R: 19090364-19106571 |
| CG7208 | chr3R: 14009378-14011437 |
| sip2 | chr2L: 6961516-6964074 |
| orb | chr3R: 19090364-19106571 |
| pigeon | chr2L: 19186043-19189938 |
| exu | chr2R: 16554924-16558379 |
| orb | chr3R: 19090364-19106571 |
| Ptpmeg | chr3L: 328097-356050 |
| CG42669 | chr3L: 2377719-2466353 |
| sip2 | chr2L: 6961516-6964074 |
| zfh1 | chr3R: 26591648-26614205 |
| CG10237 | chr2L: 19435844-19442358 |
| CG4538; Surf6 | chr3R: 15724548-15729411; chr3R: 15729384-15730589 |
| capu | chr2L: 3872658-3902860 |
| Imp | chrX: 10681902-10716815 |
| CG6145; CG33156 | chr2R: 9424893-9435082; chr2R: 9434650-9440666 |
| CG4998 | chr3L: 16332103-16338341 |
| Eip75B | chr3L: 17944053-18057796 |
| CG15891; CG15892 | chrX: 6187137-6189148 |
| Pof | chr2R: 20556901-20559041 |
| cnn | chr2R: 9326819-9337980 |
| mei-217; mei-218 | chrX: 17030611-17037023; chrX: 17030018-17037029 |
| CG4538; Surf6 | chr3R: 15724548-15729411; chr3R: 15729384-15730589 |
| CG14006 | chr2L: 5726415-5728811 |
| CG8368 | chr3L: 6596784-6601839 |
| Hr46 | chr2R: 6091617-6124853 |
| RpS28-like; CG3769 | chr2L: 9436804-9437227; chr2L: 9435399-9436842 |
| fru | chr3R: 14239995-14371308 |
| CG32392 | chr3L: 6749729-6756346 |
| Rtnl1 | chr2L: 4992808-5009720 |
| Hsp67Bb; Hsp22 | chr3L: 9365822-9368064; chr3L: 9366031-9368064 |
| mud | chrX: 14141765-14152628 |
| dpr17 | chr3R: 7920600-7937144 |
| bun | chr2L: 12455540-12546630 |
| Rootletin | chr3R: 19939317-19953915 |
| CG43954; Lasp | chr3L: 16670667-16704777; chr3L: 16669506-16704777 |
| Rtnl1 | chr2L: 4992808-5009720 |
| mei-217; mei-218 | chrX: 17030611-17037023; chrX: 17030018-17037029 |
| exu | chr2R: 16554924-16558379 |
| exu | chr2R: 16554924-16558379 |
| cnn | chr2R: 9326819-9337980 |
| cnn | chr2R: 9326819-9337980 |
| MCPH1 | chr2R: 7783499-7791429 |
| CG43340 | chr2R: 3510747-3539292 |
| chn | chr2R: 11002762-11032288 |
| lectin-46Cb | chr2R: 5700867-5704105 |
| Rtnl1 | chr2L: 4992808-5009720 |
| CG5327; CG5323 | chr2R: 14503493-14505823; chr2R: 14502587-14503504 |
| sdt | chrX: 8072582-8134507 |
| CG13380; CG4174 | chr3L: 18593250-18611043 |
| Pdp1 | chr3L: 7805015-7860469 |
| CG5327; CG5323 | chr2R: 14503493-14505823; chr2R: 14502587-14503504 |
| Tyler; Shawn | chrX: 19505559-19509650 |
| CG5327; CG5323 | chr2R: 14503493-14505823; chr2R: 14502587-14503504 |
| Rootletin | chr3R: 19939317-19953915 |
| CG12201 | chr3R: 7797416-7798627 |
| CG14183; CG14184 | chr3L: 20020919-20022120; chr3L: 20017787-20020935 |
| CG6928 | chr3L: 11996255-12001151 |
| sdt | chrX: 8072582-8134507 |
| CG42336; CG18336 | chr2R: 7182041-7185041; chr2R: 7181281-7182091 |
| Msi; Adgf-A | chr3L: 17748036-17757258; chr3L: 17744831-17757258 |
| Jafrac2 | chr3L: 3042359-3044174 |
| Mbs | chr3L: 16045059-16075126 |
| Mur2B; snmRNA: 400 | chrX: 1417587-1512188; chrX: 1452382-1452433 |
| dlg1 | chrX: 11263698-11303809 |
| CG14183; CG14184 | chr3L: 20020919-20022120; chr3L: 20017787-20020935 |
| CG42675 | chr3R: 1639544-1644545 |
| grsm | chr3R: 8775764-8793434 |
| dlg1 | chrX: 11263698-11303809 |
| dlg1 | chrX: 11263698-11303809 |
| CG13741; CG8080 | chr2R: 4980008-4982173; chr2R: 4977279-4980474 |
| CG14183; CG14184 | chr3L: 20020919-20022120; chr3L: 20017787-20020935 |
| Msi; Adgf-A | chr3L: 17748036-17757258; chr3L: 17744831-17757258 |
| cnn | chr2R: 9326819-9337980 |
| CG14183; CG14184 | chr3L: 20020919-20022120; chr3L: 20017787-20020935 |
| sky; CG43739 | chr2L: 20871335-20918931 |
| cnn | chr2R: 9326819-9337980 |
| Msi; Adgf-A | chr3L: 17748036-17757258; chr3L: 17744831-17757258 |
| lola | chr2R: 6369712-6430794 |
| toc | chr2L: 3068345-3144613 |
| CG30460 | chr2R: 12953476-12974747 |
| CG14183; CG14184 | chr3L: 20020919-20022120; chr3L: 20017787-20020935 |
| sle | chr3R: 6158893-6163995 |
| CG32700 | chrX: 9400744-9445262 |
| ewg | chrX: 162542-173751 |
| bmm | chr3L: 14769596-14779512 |
| CG17838 | chr3R: 16582451-16631963 |
| Mur89F | chr3R: 12979242-12985670 |
| CG17687 | chr3L: 13342265-13348040 |
| CG17838 | chr3R: 16582451-16631963 |
| Msi; Adgf-A | chr3L: 17748036-17757258; chr3L: 17744831-17757258 |
| CG32686 | chrX: 10265515-10269349 |
| CG12783; CG10340 | chr3R: 12437141-12439122; chr3R: 12439120-12440113 |
| CG17118; CG6443 | chr2L: 10728975-10730302; chr2L: 10730255-10731356 |
| CG10365 | chr3R: 19549086-19557227 |
| CG14995 | chr3L: 4094343-4103256 |
| Nedd4 | chr3L: 17523181-17539760 |
| mrj | chr2R: 12090834-12109298 |
| Sec16 | chrX: 12490080-12503943 |
| Dhc16F | chrX: 17958635-17972505 |
| toc | chr2L: 3068345-3144613 |
| CG12783; CG10340 | chr3R: 12437141-12439122; chr3R: 12439120-12440113 |
| CG42336; CG18336 | chr2R: 7182041-7185041; chr2R: 7181281-7182091 |
| CG13741; CG8080 | chr2R: 4980008-4982173; chr2R: 4977279-4980474 |
| RhoGEF3 | chr3L: 277437-305292 |
| CG17838 | chr3R: 16582451-16631963 |
| CG30460 | chr2R: 12953476-12974747 |
| CG8043; CG11755 | chr3R: 4502639-4503955; chr3R: 4503865-4504677 |
| CG17118; CG6443 | chr2L: 10728975-10730302; chr2L: 10730255-10731356 |
| CG12913; CG12914 | chr2R: 6135319-6136909; chr2R: 6136762-6142479 |
| xmas-2; xmas-1 | chrX: 17048143-17053339; chrX: 17045264-17048625 |
| CG10365 | chr3R: 19549086-19557227 |
| Ect4 | chr3L: 8056974-8101936 |
| xmas-2; xmas-1 | chrX: 17048143-17053339; chrX: 17045264-17048625 |
| CG43340 | chr2R: 3510747-3539292 |
| Ect4 | chr3L: 8056974-8101936 |
| CG12913; CG12914 | chr2R: 6135319-6136909; chr2R: 6136762-6142479 |
| CG10934; CG10933 | chr2R: 13609844-13610886; chr2R: 13610818-13612561 |
| sdt | chrX: 8072582-8134507 |
| Btk29A | chr2L: 8258755-8301079 |
| Jupiter | chr3R: 7416108-7445628 |
| isopeptidase-T-3 | chr2R: 15558124-15560982 |
| CG30497 | chr2R: 3624524-3670142 |
| msi | chr3R: 21341840-21434145 |
| mtd | chr3R: 1095838-1176165 |
| CG13741; CG8080 | chr2R: 4980008-4982173; chr2R: 4977279-4980474 |
| CG17838 | chr3R: 16582451-16631963 |
| CG10962; CR43836 | chrX: 8866946-8943024; chrX: 8866940-8868288 |
| CG4390 | chr3R: 15882140-15885487 |
| CG16758 | chr3L: 2499372-2503606 |
| CG10332; IM18 | chr2R: 19488437-19489296 |
| sesB; Ant2 | chrX: 10674926-10680940; chrX: 10672987-10680913 |
| rumi; CG31139 | chr3R: 18573412-18574778; chr3R: 18574736-18576283 |
| cdc14 | chr2L: 7802415-7810697 |
| MCPH1 | chr2R: 7783499-7791429 |
| Jupiter | chr3R: 7416108-7445628 |
| CG34315; lat | chr2R: 9090233-9094027 |
| CG33791; CG18170 | chr3L: 1602954-1617355 |
| CG13921 | chr3L: 1746907-1752390 |
| Dif; dl | chr2L: 17436830-17450360; chr2L: 17413248-17439923 |
| CG12207 | chr3R: 10114794-10127998 |
| koko | chr3R: 14222717-14230448 |
| RhoGEF3 | chr3L: 277437-305292 |
| sm | chr2R: 15405369-15519013 |
| CG31016 | chr3R: 26431911-26436178 |
| CG33523 | chr3L: 5917153-5923220 |
| CG17494 | chr2L: 22471804-22478575 |
| Cul-3 | chr2L: 15265245-15272024 |
| inaE | chrX: 13677782-13705526 |
| rumi; CG31139 | chr3R: 18573412-18574778; chr3R: 18574736-18576283 |
| Jupiter | chr3R: 7416108-7445628 |
| CG17838 | chr3R: 16582451-16631963 |
| Alh | chr3R: 2920318-2949907 |
| CG7777 | chr2R: 7323751-7329720 |
| CG33722; CG18749 | chr3R: 4069781-4073577 |
| tipE; CG18675 | chr3L: 4173884-4192968; chr3L: 4188499-4193788 |
| yuri | chr2L: 15257267-15264690 |
| primo-2; primo-1 | chr3R: 9535748-9537557 |
| CG31523 | chr3R: 291139-304828 |
| bol | chr3L: 9091208-9123452 |
| CG34315; lat | chr2R: 9090233-9094027 |
| CG30427 | chr2R: 20816270-20824994 |
| CG10089 | chr3L: 13458909-13467643 |
| CG42360 | chr2R: 20494812-20498049 |
| CG4095; I(1)G0255 | chrX: 6575788-6577729; chrX: 6572499-6575887 |
| Hsc70-4 | chr3R: 11068370-11072336 |
| CG10089 | chr3L: 13458909-13467643 |
| toc | chr2L: 3068345-3144613 |
| tacc | chr3R: 559376-574778 |
| CG4095; I(1)G0255 | chrX: 6575788-6577729; chrX: 6572499-6575887 |
| CG43462 | chr3R: 4190050-4303315 |
| primo-2; primo-1 | chr3R: 9535748-9537557 |
| CG17378 | chr2L: 6774041-6778444 |
| dikar | chr3L: 6721900-6736264 |
| Sac1 | chr3L: 1247817-1250451 |
| dnc | chrX: 3070473-3237800 |
| CG32473 | chr3R: 9128375-9138507 |
| larp | chr3R: 24143905-24162162 |
| Tyler; Shawn | chrX: 19505559-19509650 |
| CG4662 | chr3R: 15673045-15680125 |
| rumi; CG31139 | chr3R: 18573412-18574778; chr3R: 18574736-18576283 |
| CG4095; I(1)G0255 | chrX: 6575788-6577729; chrX: 6572499-6575887 |
| CG6136 | chr3R: 11195758-11197934 |
| scrib | chr3R: 22362072-22429773 |
| CG34315; lat | chr2R: 9090233-9094027 |
| Pop2 | chr3L: 11991498-11996015 |
| Tyler; Shawn | chrX: 19505559-19509650 |
| heph | chr3R: 27672709-27819000 |
| sec8; CG2082 | chr3R: 1580654-1584699; chr3R: 1584402-1606798 |
| Nedd4 | chr3L: 17523181-17539760 |
| sec8; CG2082 | chr3R: 1580654-1584699; chr3R: 1584402-1606798 |
| Glycogenin | chr2R: 17087988-17100474 |
| chif; CG42231 | chr2L: 16342430-16352600; chr2L: 16342426-16352600 |
| CG18135; CG3808 | chr3L: 18983126-18991383; chr3L: 18990975-18993708 |
| Syx1A; 4EHP | chr3R: 19889308-19934671; chr3R: 19927322-19931465 |
| exba | chr3R: 1421086-1426858 |
| sec8; CG2082 | chr3R: 1580654-1584699; chr3R: 1584402-1606798 |
| sec8; CG2082 | chr3R: 1580654-1584699; chr3R: 1584402-1606798 |
| Tyler; Shawn | chrX: 19505559-19509650 |
| CG7656 | chr3L: 15578531-15581275 |
| CG43462 | chr3R: 4190050-4303315 |
| CG33722; CG18749 | chr3R: 4069781-4073577 |
| loqs | chr2L: 13382648-13385671 |
| CG13741; CG8080 | chr2R: 4980008-4982173; chr2R: 4977279-4980474 |
| dlt; alpha-Spec | chr3L: 1789631-1795328; chr3L: 1778485-1795328 |
| CG43347; CG1628 | chrX: 10492179-10496747; chrX: 10496674-10514223 |
| sec8; CG2082 | chr3R: 1580654-1584699; chr3R: 1584402-1606798 |
| sec8; CG2082 | chr3R: 1580654-1584699; chr3R: 1584402-1606798 |
| capu | chr2L: 3872658-3902860 |
| CLIP-190 | chr2L: 17384700-17409698 |
| CG43954; Lasp | chr3L: 16670667-16704777; chr3L: 16669506-16704777 |
| CG31473; CG31472 | chr3R: 3636254-3637634; chr3R: 3637536-3638731 |
| mus201; Chrac-14 | chr2L: 8437411-8442352 |
| Nedd4 | chr3L: 17523181-17539760 |
| CG42588 | chr3L: 12807354-12817750 |
| mir-4963; CG42699; | chrX: 5684786-5684894; chrX: 5658608-5683609; |
| CG15767 | chrX: 5683520-5684952 |
| gish | chr3R: 12098176-12128443 |
| CG3800 | chr2R: 18822092-18824453 |
| Ubi-p63E | chr3L: 3899259-3903184 |
| UbcD2 | chr2L: 10765010-10767939 |
| spir | chr2L: 20311219-20348386 |
| exba | chr3R: 1421086-1426858 |
| Stat92E | chr3R: 16361405-16378034 |
| Den1 | chr2R: 8225307-8227050 |
| Lpin; kermit | chr2R: 4024501-4044128; chr2R: 4034423-4044126 |
| CG33523 | chr3L: 5917153-5923220 |
| HtrA2 | chr3R: 10334771-10337434 |
| CG42446; CG17931 | chr3R: 12183866-12185830; chr3R: 12183332-12185040 |
| Ect4 | chr3L: 8056974-8101936 |
| CG33260; Trl; CG42507 | chr3L: 14741029-14754149; chr3L: 14741029-14751062; |
| | chr3L: 14753620-14754944 |
| CG12645 | chrX: 10047645-10049042 |
| Cyp6u1; CG30157 | chr2R: 2861543-2862638; chr2R: 2859617-2862514 |
| mge | chr3L: 3907852-3909931 |
| CG42446; CG17931 | chr3R: 12183866-12185830; chr3R: 12183332-12185040 |
| cup; CG34310 | chr2L: 6663968-6674780 |
| Cyp6u1; CG30157 | chr2R: 2861543-2862638; chr2R: 2859617-2862514 |
| CG42446; CG17931 | chr3R: 12183866-12185830; chr3R: 12183332-12185040 |
| Ggamma1 | chr2R: 4788909-4792296 |
| CG34280; CG34279; Sur-8 | chr3R: 13228084-13229010; chr3R: 13220921-13228237; |
| | chr3R: 13228994-13229440 |
| vfl | chrX: 19660978-19677682 |
| CG34280; CG34279; Sur-8 | chr3R: 13228084-13229010; chr3R: 13220921-13228237; |
| | chr3R: 13228994-13229440 |
| CG13220 | chr2R: 7171788-7172603 |
| CG12214 | chr2R: 6043421-6046436 |
| CG8892 | chr2L: 4979513-4981592 |
| Smn | chr3L: 16573498-16574647 |
| CG14962 | chr3L: 3163786-3166250 |
| CG14053 | chrX: 2085984-2087586 |
| CG14006 | chr2L: 5726415-5728811 |
| tan | chr3L: 6048236-6050102 |
| twin | chr3R: 20022615-20047687 |
| twin | chr3R: 20022615-20047687 |
| CG5877 | chrX: 15187064-15191261 |
| CG7255 | chr3L: 15297355-15304295 |
| CG18135; CG3808 | chr3L: 18983126-18991383; chr3L: 18990975-18993708 |
| CR43960; CR32657 | chrX: 11938177-12009871; chrX: 11981244-11981728 |
| FucTB | chr2L: 9428771-9431029 |
| GstD9; GstD1 | chr3R: 8191902-8193286; chr3R: 8193269-8194987 |
| CG13016 | chr2R: 10045276-10046939 |
| timeout | chr3R: 8914373-8989598 |
| sm | chr2R: 15405369-15519013 |
| bol | chr3L: 9091208-9123452 |
| CG32719 | chrX: 7402684-7406935 |
| sm | chr2R: 15405369-15519013 |
| rtGEF | chr2L: 20350436-20365254 |
| CG42676 | chr3L: 1803107-1806819 |
| Tpr2 | chr2L: 16491540-16507621 |
| Apc2 | chr3R: 19989690-19994041 |
| CoVIIc | chr2R: 5936985-5938379 |
| CG32264; ntc | chr3L: 3714826-3806459; chr3L: 3803832-3806459 |
| drongo | chr2L: 833584-851096 |
| CG1440 | chrX: 8331113-8334969 |
| CG1440 | chrX: 8331113-8334969 |
| sqd | chr3R: 9460679-9472026 |
| CG17838 | chr3R: 16582451-16631963 |
| CG31473; CG31472 | chr3R: 3636254-3637634; chr3R: 3637536-3638731 |
| CG15547; Sap-r | chr3R: 26709186-26714795; chr3R: 26707514-26709225 |
| CG33223 | chrX: 8359723-8377918 |
| mbf1 | chr3L: 16567536-16569971 |
| hts | chr2R: 15284537-15312454 |
| Efa6 | chr3R: 18413477-18434564 |
| SpdS | chr3R: 5457361-5459395 |
| CG10089 | chr3L: 13458909-13467643 |
| NnaD | chrX: 13586325-13608468 |
| Adar; CG42666 | chrX: 1667752-1747700; chrX: 1667752-1682100 |
| CG6404 | chr3L: 10877361-10879571 |
| CG7816 | chr3R: 25689423-25694618 |
| Pde1c | chr2L: 11814930-11928570 |
| Ptp10D | chrX: 11516048-11571371 |
| CG16758 | chr3L: 2499372-2503606 |
| CG31638 | chr2L: 6491122-6496986 |
| CR43960; CR32657 | chrX: 11938177-12009871; chrX: 11981244-11981728 |
| Cyp6u1; CG30157 | chr2R: 2861543-2862638; chr2R: 2859617-2862514 |
| Tsp | chr2L: 6686786-6709085 |
| CG42708 | chr2R: 8557988-8567094 |
| Ptp10D | chrX: 11516048-11571371 |
| PR2 | chr2R: 9030339-9040182 |
| dre4 | chr3L: 1871574-1876312 |
| Cyp6u1; CG30157 | chr2R: 2861543-2862638; chr2R: 2859617-2862514 |
| CG33722; CG18749 | chr3R: 4069781-4073577 |
| CG17118; CG6443 | chr2L: 10728975-10730302; chr2L: 10730255-10731356 |
| CG15547; Sap-r | chr3R: 26709186-26714795; chr3R: 26707514-26709225 |
| Akap200 | chr2L: 8415690-8430984 |
| Alh | chr3R: 2920318-2949907 |
| Taf4 | chr3L: 16106312-16114751 |
| CG13358; CG13359 | chrX: 697457-701337; chrX: 695702-697503 |
| Cbl | chr3L: 8418054-8425346 |
| CG8176; JHDM2 | chr3R: 5341758-5352964; chr3R: 5341731-5346660 |
| CG33260; Trl; CG42507 | chr3L: 14741029-14754149; chr3L: 14741029-14751062; |
| | chr3L: 14753620-14754944 |
| f-cup | chr3R: 9520979-9526560 |
| CG10089 | chr3L: 13458909-13467643 |
| Aats-trp | chr3R: 5089539-5092670 |
| CG42321 | chr2R: 9302483-9326550 |
| CG3800 | chr2R: 18822092-18824453 |
| CG16758 | chr3L: 2499372-2503606 |
| Su(var)2-10 | chr2R: 5003631-5009507 |
| CG31875; SoYb | chr2L: 9996932-10002736; chr2L: 9995561-10002446 |
| E(bx) | chr3L: 233926-246912 |
| Alh | chr3R: 2920318-2949907 |
| Jafrac2 | chr3L: 3042359-3044174 |
| CG15891; CG15892 | chrX: 6187137-6189148 |
| ovo | chrX: 4942330-4964962 |
| Cka | chr2L: 8030721-8040826 |
| yuri | chr2L: 15257267-15264690 |
| dsx | chr3R: 3750045-3793130 |
| Tsp | chr2L: 6686786-6709085 |
| CG18135; CG3808 | chr3L: 18983126-18991383; chr3L: 18990975-18993708 |
| ldh | chr3L: 8349504-8354006 |
| CG14995 | chr3L: 4094343-4103256 |
| yuri | chr2L: 15257267-15264690 |
| CG1513 | chr2R: 5770077-5779194 |
| CG43253; CG34253; CR32194 | chr3L: 18078405-18080158; chr3L: 18079774-18080420; |
| | chr3L: 18074221-18078410 |
| aPKC | chr2R: 10831963-10850451 |
| NnaD | chrX: 13586325-13608468 |
| Smn | chr3L: 16573498-16574647 |
| Fs | chr2R: 11111061-11129084 |
| Klp10A | chrX: 11023395-11031056 |
| CG42512; CG32573 | chrX: 16545409-16547308 |
| Vrp1 | chr2R: 17994243-18010053 |
| CG17838 | chr3R: 16582451-16631963 |
| CG30499 | chr2R: 3451939-3453334 |
| Tim10; CG42497 | chr2R: 17577720-17578643 |
| CG15891; CG15892 | chrX: 6187137-6189148 |
| CG42380; CG9865; CG42379; | chr2R: 17555046-17557560 |
| CG42381 | |
| CG4266 | chr2R: 17049218-17056163 |
| CG6091 | chr3L: 11598536-11602540 |
| cl | chr2L: 5520135-5521382 |
| aop | chr2L: 2156484-2178749 |
| Atet | chr2L: 4333926-4345776 |
| Ckllbeta | chrX: 11686566-11695620 |
| Act87E | chr3R: 9251707-9253811 |
| exu | chr2R: 16554924-16558379 |
| elF-1A | chr3R: 14100387-14108206 |
| AIF | chr2L: 2151741-2155389 |
| yuri | chr2L: 15257267-15264690 |
| CG11475; CG11474 | chr2R: 17957230-17958791; chr2R: 17958739-17960948 |
| CR43960; CR32657 | chrX: 11938177-12009871; chrX: 11981244-11981728 |
| CG12004 | chr3L: 1569181-1575620 |
| Su(var)2-10 | chr2R: 5003631-5009507 |
| tra2 | chr2R: 10489509-10491857 |
| hang | chrX: 16316877-16331531 |
| CG15547; Sap-r | chr3R: 26709186-26714795; chr3R: 26707514-26709225 |
| simj | chr3L: 10657839-10683834 |
| CG42669 | chr3L: 2377719-2466353 |
| CG6923 | chr3R: 7570205-7575751 |
| AIF | chr2L: 2151741-2155389 |
| inaE | chrX: 13677782-13705526 |
| CG31523 | chr3R: 291139-304828 |
| CG17838 | chr3R: 16582451-16631963 |
| Mur89F | chr3R: 12979242-12985670 |
| Dhod | chr3R: 4477113-4478638 |
| Pl31 | chr2R: 7854848-7857591 |
| alph | chr3R: 25417542-25442879 |
| CG42321 | chr2R: 9302483-9326550 |
| CG6745; CG6765 | chr3L: 8505867-8511838; chr3L: 8503103-8506107 |
| CG8786 | chr3L: 19630868-19635916 |
| lig | chr2R: 3955460-3966320 |
| CG17230 | chr3R: 7263507-7291360 |
| Sfmbt | chr2L: 13169649-13176785 |
| Pop2 | chr3L: 11991498-11996015 |
| CG42351 | chr2R: 14644579-14648587 |
| I(2)03709 | chr2R: 14706972-14709755 |
| PGRP-LD; Pmi | chr3L: 5773151-5777785 |
| Fer2LCH | chr3R: 26213526-26216306 |
| CG1812 | chrX: 20382574-20386859 |
| CG42351 | chr2R: 14644579-14648587 |
| CG6745; CG6765 | chr3L: 8505867-8511838; chr3L: 8503103-8506107 |
| sick | chr2L: 19796365-19958424 |
| I(1)G0196; Cp110 | chrX: 21884237-21889294; chrX: 21889016-21909502 |
| CG9449 | chr3L: 19485153-19490823 |
| Pi3K59F; CG30184 | chr2R: 19447961-19451558; chr2R: 19451552-19452303 |
| CG34315; lat | chr2R: 9090233-9094027 |
| CG30460 | chr2R: 12953476-12974747 |
| CG14017; GluRIIA | chr2L: 5553897-5555137; chr2L: 5555074-5558994 |
| Imp | chrX: 10681902-10716815 |
| rn | chr3R: 3099460-3135119 |
| CG5315 | chr3R: 18369415-18373567 |
| zip | chr2R: 20878093-20899488 |
| CG1882 | chr2R: 3700011-3702250 |
| CG33523 | chr3L: 5917153-5923220 |
| sgg | chrX: 2527985-2571879 |
| Pif1A; Pif1B; CG33191 | chr3R: 4604101-4605645; chr3R: 4600008-4626898 |
| garz | chr2R: 8214179-8221324 |
| CG4300 | chr3L: 12264756-12270351 |
| CG13029 | chr3L: 16853722-16854940 |
| Rcd-1 | chrX: 19532913-19534784 |
| CG12948; CG34409 | chr3R: 5571889-5573254; chr3R: 5568599-5572089 |
| CG3339 | chr3R: 23120267-23148047 |
| Dap160 | chr2L: 21134439-21142347 |
| Rcd1 | chr2R: 10035768-10040624 |
| Sxl; mir-4956 | chrX: 6968583-6992089; chrX: 6979770-6979886 |
| I(2)37Cc | chr2L: 19122040-19123869 |
| Adf1 | chr2R: 2549775-2555590 |
| yuri | chr2L: 15257267-15264690 |
| yin | chrX: 3756996-3762359 |
| Tpi | chr3R: 25958698-25960321 |
| sec8; CG2082 | chr3R: 1580654-1584699; chr3R: 1584402-1606798 |
| CG13349 | chr2R: 9846033-9847909 |
| CG14183; CG14184 | chr3L: 20020919-20022120; chr3L: 20017787-20020935 |
| eRF1; CG5618 | chr3L: 20341455-20346973; chr3L: 20346549-20349732 |
| CG16833 | chr2L: 10989305-10997700 |
| sip2 | chr2L: 6961516-6964074 |
| pum | chr3R: 4892065-5063404 |
| CR42859; CG31686 | chr2L: 2245230-2246207; chr2L: 2244637-2245352 |
| CG8086 | chr2L: 8244160-8257502 |
| CG7927 | chr3L: 8103468-8106420 |
| koko | chr3R: 14222717-14230448 |
| capu | chr2L: 3872658-3902860 |
| ArfGAP3 | chr3L: 22707413-22710677 |
| slgA | chrX: 21245018-21256188 |
| rin | chr3R: 9472726-9480285 |
| prtp | chrX: 11610821-11613927 |
| vfl | chrX: 19660978-19677682 |
| elF-3p40 | chr2L: 5051193-5053094 |
| CG14183; CG14184 | chr3L: 20020919-20022120; chr3L: 20017787-20020935 |
| Girdin; CG42494 | chr3L: 3178930-3185287; chr3L: 3184404-3186549 |
| CG10320 | chr2R: 17545580-17546282 |
| Pl31 | chr2R: 7854848-7857591 |
| Msi; Adgf-A | chr3L: 17748036-17757258; chr3L: 17744831-17757258 |
| CG4095; I(1)G0255 | chrX: 6575788-6577729; chrX: 6572499-6575887 |
| CG30460 | chr2R: 12953476-12974747 |
| skd | chr3L: 20985914-21020534 |
| CG14183; CG14184 | chr3L: 20020919-20022120; chr3L: 20017787-20020935 |
| rab3-GAP | chr2L: 12058304-12063171 |
| MCPH1 | chr2R: 7783499-7791429 |
| CG42855; Sik3 | chr2R: 14575637-14593310; chr2R: 14574877-14576146 |
| Mical | chr3R: 5827276-5868617 |
| CG13220 | chr2R: 7171788-7172603 |
| Pgd | chrX: 2039169-2042599 |
| vlc | chr2R: 1589605-1592944 |
| Adk3 | chr3R: 6713288-6715310 |
| Prosalpha5 | chr2R: 13300269-13301274 |
| E(bx) | chr3L: 233926-246912 |
| Msi; Adgf-A | chr3L: 17748036-17757258; chr3L: 17744831-17757258 |
| Mitf | chr4: 1219484-1228244 |
| CG10365 | chr3R: 19549086-19557227 |
| smg | chr3L: 8983417-8991089 |
| CG13921 | chr3L: 1746907-1752390 |
| CG34294; tau | chr3R: 23466365-23482718; chr3R: 23465608-23466517 |
| UbcD2 | chr2L: 10765010-10767939 |
| Girdin; CG42494 | chr3L: 3178930-3185287; chr3L: 3184404-3186549 |
| CG5794 | chr3R: 20461091-20475765 |
| cnc | chr3R: 19011302-19052430 |
| CG1773; CG10459 | chr2R: 5595076-5596227; chr2R: 5593789-5595085 |
| brat | chr2L: 19133816-19179758 |
| sle | chr3R: 6158893-6163995 |
| RhoGEF3 | chr3L: 277437-305292 |
| CG17838 | chr3R: 16582451-16631963 |
| rdx | chr3R: 9793674-9857777 |
| f-cup | chr3R: 9520979-9526560 |
| CG14183; CG14184 | chr3L: 20020919-20022120; chr3L: 20017787-20020935 |
| sesB; Ant2 | chrX: 10674926-10680940; chrX: 10672987-10680913 |
| CG1140 | chr3L: 1963143-1966805 |
| Msi; Adgf-A | chr3L: 17748036-17757258; chr3L: 17744831-17757258 |
| lqf | chr3L: 7521880-7533112 |
| CG32112 | chr3L: 12734818-12738254 |
| NnaD | chrX: 13586325-13608468 |
| Girdin; CG42494 | chr3L: 3178930-3185287; chr3L: 3184404-3186549 |
| CG42336; CG18336 | chr2R: 7182041-7185041; chr2R: 7181281-7182091 |
| Snp | chr2R: 17948458-17957262 |
| CG9576 | chrX: 20054388-20059863 |
| wech | chr2R: 3366358-3377392 |
| eff | chr3R: 10558427-10566933 |
| CG10934; CG10933 | chr2R: 13609844-13610886; chr2R: 13610818-13612561 |
| pum | chr3R: 4892065-5063404 |
| sesB; Ant2 | chrX: 10674926-10680940; chrX: 10672987-10680913 |
| prominin-like | chr3L: 3114447-3127724 |
| ldh | chr3L: 8349504-8354006 |
| Hmgcr | chr3R: 19559046-19576868 |
| CG7220 | chr2R: 6618861-6626116 |
| CG14183; CG14184 | chr3L: 20020919-20022120; chr3L: 20017787-20020935 |
| vib | chr3R: 15041156-15052373 |
| Dgk | chr2R: 3722317-3773487 |
| CG12207 | chr3R: 10114794-10127998 |
| RanGap | chr2L: 19442041-19447322 |
| aret | chr2L: 12175004-12313438 |
| Jafrac2 | chr3L: 3042359-3044174 |
| CG32425 | chr3L: 20490423-20507207 |
| chic | chr2L: 5972115-5981018 |
| elF5B | chr3L: 3428658-3460977 |
| CG33054; CG33056 | chr3L: 21191361-21196332; chr3L: 21190511-21195982 |
| CG7616 | chr3L: 11162917-11165511 |
| cnn | chr2R: 9326819-9337980 |
| rab3-GAP | chr2L: 12058304-12063171 |
| sno | chrX: 13089532-13105699 |
| CG3967 | chr3L: 9406696-9419777 |
| CG33170 | chr3L: 22857700-22860635 |
| Taz | chr2R: 8640531-8643915 |
| CG3499 | chr2R: 18770499-18773770 |
| CG17724; Kdm4B; seq | chr2R: 9063599-9085869; chr2R: 9063599-9073317; |
| | 9chr2R: 9066673-077579 |
| dUTPase | chr2L: 11011293-11012135 |
| Calx | chr3R: 16803907-16841335 |
| CG4095; I(1)G0255 | chrX: 6575788-6577729; chrX: 6572499-6575887 |
| Pi4KIIalpha | chr3R: 1383385-1389347 |
| CG7946 | chr3R: 25878236-25880419 |
| Msi; Adgf-A | chr3L: 17748036-17757258; chr3L: 17744831-17757258 |
| sesB; Ant2 | chrX: 10674926-10680940; chrX: 10672987-10680913 |
| GlcAT-S | chr2L: 9616469-9623109 |
| Pka-R2 | chr2R: 5881395-5913547 |
| cnn | chr2R: 9326819-9337980 |
| CG14183; CG14184 | chr3L: 20020919-20022120; chr3L: 20017787-20020935 |
| aret | chr2L: 12175004-12313438 |
| sesB; Ant2 | chrX: 10674926-10680940; chrX: 10672987-10680913 |
| CG42668 | chr3R: 16135066-16149918 |
| Bl-1 | chr3L: 8300245-8302041 |
| Sgt | chr2L: 17471603-17474773 |
| emp | chr2R: 20863979-20872321 |
| CG3192 | chrX: 6571294-6573151 |
| Girdin; CG42494 | chr3L: 3178930-3185287; chr3L: 3184404-3186549 |
| CG17018 | chr2L: 22311931-22368796 |
| lok | chr2L: 20061477-20064381 |
| mex1 | chr3L: 15510524-15512692 |
| CG14562; CG7448 | chr3L: 21942925-21944650; chr3L: 21935321-21942966 |
| Alr; CG32528 | chrX: 19642417-19644204; chrX: 19640090-19642491 |
| Girdin; CG42494 | chr3L: 3178930-3185287; chr3L: 3184404-3186549 |
| CG10512 | chr3L: 21196020-21201192 |
| Lin29 | chr4: 396266-416428 |
| fz3 | chrX: 664061-677344 |
| CG14183; CG14184 | chr3L: 20020919-20022120; chr3L: 20017787420020935 |
| Nacalpha | chr2R: 8647198-8648377 |
| CG14183; CG14184 | chr3L: 20020919-20022120; chr3L: 20017787-20020935 |
| Den1 | chr2R: 8225307-8227050 |
| Not1 | chr2R: 5453651-5466512 |
| apt | chr2R: 19452377-19487049 |
| CG32944 | chr3R: 232949-241835 |
| app | chr3L: 12199338-12259941 |
| Fer1HCH | chr3R: 26211295-26213900 |
| CG4095; I(1)G0255 | chrX: 6575788-6577729; chrX: 6572499-6575887 |
| xmas-2; xmas-1 | chrX: 17048143-17053339; chrX: 17045264-17048625 |
| cnn | chr2R: 9326819-9337980 |
| cnn | chr2R: 9326819-9337980 |
| xmas-2; xmas-1 | chrX: 17048143-17053339; chrX: 17045264-17048625 |
| polo | chr3L: 20302754-20306566 |
| SRPK | chr2R: 11268223-11275356 |
| cnn | chr2R: 9326819-9337980 |
| CG9992 | chrX: 16275984-16278937 |
| Pif1A; Pif1B; CG33191 | chr3R: 4604101-4605645; chr3R: 4600008-4626898 |
| CG34394 | chr2L: 3358640-3373372 |
| Droj2 | chr3R: 9207799-9211013 |
| CG17494 | chr2L: 22471804-22478575 |
| coro | chr2R: 2749506-2760274 |
| Imp | chrX: 10681902-10716815 |
| CG14562; CG7448 | chr3L: 21942925-21944650; chr3L: 21935321-21942966 |
| Hsc70-4 | chr3R: 11068370-11072336 |
| DOR | chr3L: 4406943-4420648 |
| CG7192 | chrX: 17991591-17995356 |
| CG3887 | chr2L: 5010922-5012127 |
| CG42669 | chr3L: 2377719-2466353 |
| CG43674 | chr3R: 5165938-5188666 |
| Jupiter | chr3R: 7416108-7445628 |
| CG7656 | chr3L: 15578531-15581275 |
| toc | chr2L: 3068345-3144613 |
| LRP1 | chr2R: 4061670-4113943 |
| cnn | chr2R: 9326819-9337980 |
| Dhc98D | chr3R: 24312675-24345120 |
| CG4095; I(1)G0255 | chrX: 6575788-6577729; chrX: 6572499-6575887 |
| aret | chr2L: 12175004-12313438 |
| Lam | chr2L: 5542544-5546642 |
| Drep-3 | chr2R: 7940449-7950374 |
| Pif1A; Pif1B; CG33191 | chr3R: 4604101-4605645; chr3R: 4600008-4626898 |
| alph | chr3R: 25417542-25442879 |
| CG3500 | chr2R: 19243895-19245088 |
| Nedd4 | chr3L: 17523181-17539760 |
| del | chr2L: 21145137-21148546 |
| Lin29 | chr4: 396266-416428 |
| GEFmeso; CG42697 | chr2R: 14441624-14499260; chr2R: 14499150-14500235 |
| msi | chr3R: 21341840-21434145 |
| CG42336; CG18336 | chr2R: 7182041-7185041; chr2R: 7181281-7182091 |
| CG14968 | chr3L: 3299336-3303112 |
| Tyler; Shawn | chrX: 19505559-19509650 |
| CLIP-190 | chr2L: 17384700-17409698 |
| CG31279 | chr3R: 12256403-12258619 |
| Tyler; Shawn | chrX: 19505559-19509650 |
| sesB; Ant2 | chrX: 10674926-10680940; chrX: 10672987-10680913 |
| Lam | chr2L: 5542544-5546642 |
| primo-2; primo-1 | chr3R: 9535748-9537557 |
| CG42795 | chr3R: 6051476-6076999 |
| vig | chr2L: 15062931-15070316 |
| CG4095; I(1)G0255 | chrX: 6575788-6577729; chrX: 6572499-6575887 |
| GABA-B-R3 | chr2L: 750609-762142 |
| ps | chr3R: 5243664-5275190 |
| kuk | chr3R: 12905955-12910503 |
| fal | chr3L: 19287867-19291930 |
| CG6664 | chr3L: 17005986-17009280 |
| Adar; CG42666 | chrX: 1667752-1747700; chrX: 1667752-1682100 |
| RhoGEF3 | chr3L: 277437-305292 |
| Tyler; Shawn | chrX: 19505559-19509650 |
| CG12360 | chr3R: 8852747-8856372 |
| CG31473; CG31472 | chr3R: 3636254-3637634; chr3R: 3637536-3638731 |
| Prosbeta7 | chr3R: 1182734-1184086 |
| tamo | chr2R: 20014161-20021074 |
| Pka-C3 | chr3L: 15918904-15947897 |
| Droj2 | chr3R: 9207799-9211013 |
| ssh | chr3R: 20764725-20770678 |
| CG12214 | chr2R: 6043421-6046436 |
| Msi; Adgf-A | chr3L: 17748036-17757258; chr3L: 17744831-17757258 |
| CG41520 | chrU: 1242085-1369506 |
| kdn | chrX: 6244662-6255226 |
| CG31473; CG31472 | chr3R: 3636254-3637634; chr3R: 3637536-3638731 |
| snoRNA: Or-aca2; CR33222; | chrX: 7792469-7795335; chrX: 7793653-7795362; |
| RpS6 | chrX: 7795095-7795236 |
| mud | chrX: 14141765-14152628 |
| mp | chr3L: 6991943-7046528 |
| Jupiter | chr3R: 7416108-7445628 |
| CG12783; CG10340 | chr3R: 12437141-12439122; chr3R: 12439120-12440113 |
| CG10249 | chr2R: 10788980-10815930 |
| stet | chr3L: 1475438-1489547 |
| CG11526 | chr3L: 3322025-3328871 |
| CG5065 | chr2R: 12495159-12512399 |
| elF5B | chr3L: 3428658-3460977 |
| Pdk1 | chr3L: 129450-145895 |
| CG8892 | chr2L: 4979513-4981592 |
| tipE; CG18675 | chr3L: 4173884-4192968; chr3L: 4188499-4193788 |
| Got2 | chr2L: 1984512-1987788 |
| qua | chr2L: 17486806-17497203 |
| GEFmeso; CG42697 | chr2R: 14441624-14499260; chr2R: 14499150-14500235 |
| CG30497 | chr2R: 3624524-3670142 |
| fal | chr3L: 19287867-19291930 |
| nemy | chr2R: 8547002-8557722 |
| spir | chr2L: 20311219-20348386 |
| CG10565 | chr3L: 21126350-21129453 |
| ZnT35C | chr2L: 15228728-15247832 |
| CG17838 | chr3R: 16582451-16631963 |
| mud | chrX: 14141765-14152628 |
| Smc5 | chr3L: 21562276-21566623 |
| Cep97 | chr2L: 3867714-3871425 |
| chb | chr3L: 21165953-21176378 |
| glob3; CG1208 | chr3R: 1657350-1664504; chr3R: 1655784-1657507 |
| D | chr3L: 14168710-14171720 |
| CR32660; Sclp | chrX: 11814030-11814507; chrX: 11814037-11818695 |
| GEFmeso; CG42697 | chr2R: 14441624-14499260; chr2R: 14499150-14500235 |
| CG43347; CG1628 | chrX: 10492179-10496747; chrX: 10496674-10514223 |
| orb2; CG43783 | chr3L: 8925335-8947372; chr3L: 8930200-8947563 |
| toc | chr2L: 3068345-3144613 |
| wmd | chr2R: 19432342-19435603 |
| nrv2 | chr2L: 6789770-6798862 |
| CG12963 | chr2R: 11442951-11449941 |
| I(3)05822 | chr3R: 14114579-14124606 |
| fu12 | chr2L: 8449691-8463990 |
| Adar; CG42666 | chrX: 1667752-1747700; chrX: 1667752-1682100 |
| Jupiter | chr3R: 7416108-7445628 |
| CG12963 | chr2R: 11442951-11449941 |
| heph | chr3R: 27672709-27819000 |
| CG30008; CG12923 | chr2R: 5775153-5778646 |
| CG14562; CG7448 | chr3L: 21942925-21944650; chr3L: 21935321-21942966 |
| kuk | chr3R: 12905955-12910503 |
| skap | chr3R: 4764405-4768749 |
| CG7414 | chr3L: 21817840-21820956 |
| tipE; CG18675 | chr3L: 4173884-4192968; chr3L: 4188499-4193788 |
| CG17118; CG6443 | chr2L: 10728975-10730302; chr2L: 10730255-10731356 |
| cnn | chr2R: 9326819-9337980 |
| CG13004 | chrX: 16720714-16729547 |
| CG12783; CG10340 | chr3R: 12437141-12439122; chr3R: 12439120-12440113 |
| sgg | chrX: 2527985-2571879 |
| CG5327; CG5323 | chr2R: 14503493-14505823; chr2R: 14502587-14503504 |
| Tyler; Shawn | chrX: 19505559-19509650 |
| CG10326 | chr3R: 12469327-12470893 |
| CG42362; CG42363; CG42364 | chr2R: 17551938-17553613; chr2R: 17553445-17554084 |
| CG4847 | chr2R: 13399028-13401364 |
| CG34315; lat | chr2R: 9090233-9094027 |
| CG10332; IM18 | chr2R: 19488437-19489296 |
| CR32660; Sclp | chrX: 11814030-11814507; chrX: 11814037-11818695 |
| CG10332; IM18 | chr2R: 19488437-19489296 |
| Mical | chr3R: 5827276-5868617 |
| Cam; CR43900 | chr2R: 8146914-8148318; chr2R: 8146903-8166314 |
| CG1620 | chr2R: 3381311-3383971 |
| fz | chr3L: 14267443-14361739 |
| CR32660; Sclp | chrX: 11814030-11814507; chrX: 11814037-11818695 |
| Peritrophin-15b | chr2L: 8394446-8395122 |
| qtc; mir-2495 | chr2L: 5068570-5068684; chr2L: 5060815-5070750 |
| Got1 | chr2R: 12025857-12029439 |
| Psa | chr3L: 1502843-1523380 |
| exu | chr2R: 16554924-16558379 |
| CG5877 | chrX: 15187064-15191261 |
| stmA; CG30356 | chr2R: 4617003-4621809; chr2R: 4616465-4617007 |
| fal | chr3L: 19287867-19291930 |
| CR42859; CG31686 | chr2L: 2245230-2246207; chr2L: 2244637-2245352 |
| elF5B | chr3L: 3428658-3460977 |
| primo-2; primo-1 | chr3R: 9535748-9537557 |
| 14-3-3epsilon | chr3R: 14068253-14075030 |
| CG8560 | chr3L: 7395432-7397281 |
| papi | chr2L: 2220750-2225544 |
| stmA; CG30356 | chr2R: 4617003-4621809; chr2R: 4616465-4617007 |
| CG6511 | chr3L: 8714424-8719208 |
| CG12605 | chr3L: 3954251-3964771 |
| CG5327; CG5323 | chr2R: 14503493-14505823; chr2R: 14502587-14503504 |
| Bruce | chr3R: 6137757-6157075 |
| tws | chr3R: 5951851-5965494 |
| CG11739 | chr3R: 204386-206932 |
| CG14017; GluRIIA | chr2L: 5553897-5555137; chr2L: 5555074-5558994 |
| CG8001 | chr3L: 1756159-1759208 |
| CG13004 | chrX: 16720714-16729547 |
| Nipped-A | chr2R: 1065506-1138553 |
| CR42859; CG31686 | chr2L: 2245230-2246207; chr2L: 2244637-2245352 |
| SPoCk | chr3L: 22742544-22780687 |
| TER94 | chr2R: 5876664-5881064 |
| Oatp58Da; CG30278 | chr2R: 18105961-18108393; chr2R: 18105022-18106003 |
| Eb1 | chr2R: 2636732-2643805 |
| CG14394; Past1 | chr3R: 8520868-8523265; chr3R: 8523044-8530597 |
| CG34315; lat | chr2R: 9090233-9094027 |
| Tyler; Shawn | chrX: 19505559-19509650 |
| Cul-3 | chr2L: 15265245-15272024 |
| Hsp60 | chrX: 11002515-11006341 |
| Syx1A; 4EHP | chr3R: 19889308-19934671; chr3R: 19927322-19931465 |
| CG9391 | chr3L: 21259780-21261435 |
| ttv | chr2R: 10413772-10475422 |
| glob3; CG1208 | chr3R: 1657350-1664504; chr3R: 1655784-1657507 |
| CG18173 | chr3L: 1735853-1738106 |
| stmA; CG30356 | chr2R: 4617003-4621809; chr2R: 4616465-4617007 |
| capu | chr2L: 3872658-3902860 |
| CG8368 | chr3L: 6596784-6601839 |
| CG4238 | chr2L: 2009859-2030536 |
| CG8420 | chr3R: 5072371-5077153 |
| Dcp2 | chr3L: 15811834-15819523 |
| CG18769 | chr3L: 6543838-6587040 |
| CG31752; CG10600 | chr2L: 18833710-18839679; chr2L: 18839628-18841310 |
| CG31033 | chr3R: 25653003-25660823 |
| CG12355 | chr3L: 15486064-15488649 |
| fdl | chr2R: 8375253-8394841 |
| Dhc98D | chr3R: 24312675-24345120 |
| CG34315; lat | chr2R: 9090233-9094027 |
| CG14619 | chrX: 21857641-21870311 |
| Mlf | chr2R: 11823398-11827578 |
| gish | chr3R: 12098176-12128443 |
| glob3; CG1208 | chr3R: 1657350-1664504; chr3R: 1655784-1657507 |
| nrv2 | chr2L: 6789770-6798862 |
| CG14017; GluRIIA | chr2L: 5553897-5555137; chr2L: 5555074-5558994 |
| Dhc98D | chr3R: 24312675-24345120 |
| Bj1 | chr3L: 5923362-5925873 |
| CG12783; CG10340 | chr3R: 12437141-12439122; chr3R: 12439120-12440113 |
| CG34315; lat | chr2R: 9090233-9094027 |
| Adar; CG42666 | chrX: 1667752-1747700; chrX: 1667752-1682100 |
| CG14480 | chr2R: 13451845-13453357 |
| CG11357 | chr3L: 4542051-4553457 |
| CG5327; CG5323 | chr2R: 14503493-14505823; chr2R: 14502587-14503504 |
| twe | chr2L: 16259414-16261877 |
| PpD3 | chr3R: 5573597-5576822 |
| Jra | chr2R: 5983985-5986061 |
| pbl | chr3L: 7889209-7905051 |
| CG7580 | chr3L: 17467861-17469224 |
| att-ORFA; att-ORFB | chr3R: 16378356-16380131; chr3R: 16378111-16383403 |
| Mlf | chr2R: 11823398-11827578 |
| CoVIIc | chr2R: 5936985-5938379 |
| Pka-C3 | chr3L: 15918904-15947897 |
| stai | chr2L: 6100377-6124653 |
| Sac1 | chr3L: 1247817-1250451 |
| elF5 | chrX: 15881194-15886023 |
| Taf7 | chr3R: 3733055-3735445 |
| CG34252 | chr3L: 18063390-18066482 |
| CG34280; CG34279; Sur-8 | chr3R: 13228084-13229010; chr3R: 13220921-13228237; |
| | chr3R: 13228994-13229440 |
| Cdc37; CG12020 | chr3L: 1797305-1798610; chr3L: 1795392-1797307 |
| CG4390 | chr3R: 15882140-15885487 |
| Taf7 | chr3R: 3733055-3735445 |
| Alr; CG32528 | chrX: 19642417-19644204; chrX: 19640090-19642491 |
| app | chr3L: 12199338-12259941 |
| CG42446; CG17931 | chr3R: 12183866-12185830; chr3R: 12183332-12185040 |
| D1 | chr3R: 5063805-5066376 |
| gish | chr3R: 12098176-12128443 |
| CG6744 | chr3R: 7259210-7261425 |
| CG12783; CG10340 | chr3R: 12437141-12439122; chr3R: 12439120-12440113 |
| CG1516; cbx | chr2R: 5738208-5757872; chr2R: 5752015-5762577 |
| CG34280; CG34279; Sur-8 | chr3R: 13228084-13229010; chr3R: 13220921-13228237; |
| | chr3R: 13228994-13229440 |
| CG32795 | chrX: 2676939-2684600 |
| Ubi-p63E | chr3L: 3899259-3903184 |
| CG2970 | chr2R: 19836724-19839982 |
| CG8774 | chr3R: 9124569-9128056 |
| CG42797 | chrX: 9502979-9517276 |
| hdc | chr3R: 26103656-26190686 |
| CG15742; mew | chrX: 13108442-13152523; chrX: 13151425-13152528 |
| fbl6; CG18335; dare | chr2R: 7178499-7180676; chr2R: 7177193-7178541; |
| | chr2R: 7172653-7177253 |
| janA; janB | chr3R: 25863702-25864460; chr3R: 25864339-25865259 |
| CG14488; CG6370 | chr2R: 13654670-13655263; chr2R: 13652077-13654712 |
| CG14562; CG7448 | chr3L: 21942925-21944650; chr3L: 21935321-21942966 |
| CG42708 | chr2R: 8557988-8567094 |
| CG1640 | chrX: 13264639-13274193 |
| twin | chr3R: 20022615-20047687 |
| Rpn6 | chr2R: 10637671-10640415 |
| Pfk | chr2R: 5997245-6004962 |
| CG32479 | chr3L: 871899-895313 |
| CG7430 | chr3L: 17840016-17842472 |
| BicC | chr2L: 16042032-16048626 |
| CG5787 | chr2L: 12708792-12712952 |
| CG6404 | chr3L: 10877361-10879571 |
| Pfk | chr2R: 5997245-6004962 |
| orb | chr3R: 19090364-19106571 |
| CG15742; mew | chrX: 13108442-13152523; chrX: 13151425-13152528 |
| CG7580 | chr3L: 17467861-17469224 |
| Tom20 | chr3L: 19993744-19995546 |
| Adar; CG42666 | chrX: 1667752-1747700; chrX: 1667752-1682100 |
| Imp | chrX: 10681902-10716815 |
| grsm | chr3R: 8775764-8793434 |
| CG42855; Sik3 | chr2R: 14575637-14593310; chr2R: 14574877-14576146 |
| Trc8 | chr3R: 25324747-25329519 |
| janA; janB | chr3R: 25863702-25864460; chr3R: 25864339-25865259 |
| grsm | chr3R: 8775764-8793434 |
| CG42446; CG17931 | chr3R: 12183866-12185830; chr3R: 12183332-12185040 |
| CG2051 | chr3R: 1613059-1615106 |
| CG14488; CG6370 | chr2R: 13654670-13655263; chr2R: 13652077-13654712 |
| Cdc37; CG12020 | chr3L: 1797305-1798610; chr3L: 1795392-1797307 |
| toc | chr2L: 3068345-3144613 |
| pAbp | chr2R: 14027583-14034696 |
| CG14562; CG7448 | chr3L: 21942925-21944650; chr3L: 21935321-21942966 |
| mir-4963; CG42699; | chrX: 5684786-5684894; chrX: 5658608-5683609; |
| CG15767 | chrX: 5683520-5684952 |
| CG6084 | chr3L: 11613635-11617481 |
| Imp | chrX: 10681902-10716815 |
| orb | chr3R: 19090364-19106571 |
| Imp | chrX: 10681902-10716815 |
| CG42855; Sik3 | chr2R: 14575637-14593310; chr2R: 14574877-14576146 |
| rumi; CG31139 | chr3R: 18573412-18574778; chr3R: 18574736-18576283 |
| rumi; CG31139 | chr3R: 18573412-18574778; chr3R: 18574736-18576283 |
| salr | chr2L: 11358873-11373637 |
| CLS | chr3R: 21510958-21513092 |
| nesd | chr2L: 20093972-20096253 |
| LIMK1 | chrX: 12478418-12486226 |
| CG43679 | chr3L: 15043952-15044435 |
| Pfk | chr2R: 5997245-6004962 |
| primo-2; primo-1 | chr3R: 9535748-9537557 |
| CG8478 | chr3R: 5589372-5591857 |
| CG4230 | chr2L: 5096227-5099294 |
| CG42446; CG17931 | chr3R: 12183866-12185830; chr3R: 12183332-12185040 |
| CG7580 | chr3L: 17467861-17469224 |
| kto | chr3L: 19829714-19838044 |
| elF3-S9 | chr2R: 13423718-13426647 |
| Oatp30B | chr2L: 9521214-9540060 |
| CG9576 | chrX: 20054388-20059863 |
| CG8036 | chr3R: 4493726-4499453 |
| CG6084 | chr3L: 11613635-11617481 |
| CG34280; CG34279; Sur-8 | chr3R: 13228084-13229010; chr3R: 13220921-13228237; |
| | chr3R: 13228994-13229440 |
| Tom40 | chrX: 7630926-7634011 |
| Tom40 | chrX: 7630926-7634011 |

In some embodiments, any one or more of the above noted sex-specific introns (in Table 0.1) can be used (identified by gene name and coordinates). While the above list is made in reference to Drosophilia, in other embodiments, any of the above genes that is conserved in the other organism gene can be used for the other organism. In some embodiments, the other organism is any of those provided herein.

In some embodiments, the intron is positioned within the gene of the protective protein so as to produce only non-functional protective proteins. In some embodiments, this can be done based on the specifics of the protective protein (interesting the intron in areas known to disrupt the functionality/expression of the protein). In some embodiments, the intron can be placed after the first 25% of the gene and before the last 75% of the gene. Thus, in some embodiments, the nonnative intron is positioned between 25-75% of the gene, for example positioned at approximately 30%, 40%, 50%, 60%, or 70% of the gene encoding the protective protein.

Following excision of the sex-specific intron, a protective protein can be produced that confers resistance to a positive selection event in sex-specific manner (in some embodiments, this is because only a single sex of the organism will produce an adequate amount of the protective protein). Thus, in some embodiments, the addition of a selection event to a population of insects having the arrangement described, results in all of the females dying, as none of the females have the machinery required to remove the intron to allow for proper production of the protective protein, while more of the males would survive (as they would have had the machinery required to excise the intron). In some embodiments, the intron is excised and a protective protein (that is, a functional protein that confers a survival benefit to the organism in regard to the denoted selection event) is produce only in males. In some embodiments, the intron is excised and a protective protein is produce only in females.

### Genetically Engineered Insect

In some embodiments, a genetically engineered insect is provided. The insect can comprise a nonnative gene that encodes for a protective protein. The nonnative gene comprises an intron that is not native to the gene encoding the protective protein. The intron is excisable by splicing in a sex-specific manner (meaning that excision occurs successfully in one sex of the organism over the other sex of the organism). Excision of the intron allows the protective protein to be expressed in a sex-specific manner. Expression of the protective protein confers resistance to a selection event. Thus, such individuals can be raised in the absence of any additional environmental influences (such as a negative selection molecule), and can be rapidly and effectively concentrated down when in a mixed population (male and female, both including the noted gene) to a single sex by a selection event.

In some embodiments genetically engineered insects that comprise a sex-specific selection element are provided. The sex-specific selection element comprises an antibiotic resistance gene comprising a sex-specific intron. The intron is excisable by splicing in a sex-specific manner. Excision of the sex-specific intron allows the antibiotic gene to be expressed in a sex-specific manner. Expression of the antibiotic resistance gene confers resistance to a positive sex-specific selection.

In some embodiments, a genetically engineered insect comprises a sex-specific selection element, the sex-specific selection element comprises an antibiotic resistance gene comprising a sex-specific intron. The intron is excisable by splicing in a sex-specific manner. The excision of the sex-specific intron allows the antibiotic gene to be expressed in a sex-specific manner, and expression of the antibiotic resistance gene confers resistance to a positive sex-specific selection event. In some embodiments, the excision of the sex-specific intron occurs only in males. In some embodiments, the excision of the sex-specific intron occurs only in females.

In some embodiments, the antibiotic resistance gene is expressed only in males and confers resistance to neomycin. In some embodiments, the antibiotic resistance gene is expressed only in males and confers resistance to puromycin. In some embodiments, the antibiotic resistance gene is expressed only in females and confers resistance to neomycin. In some embodiments, the antibiotic resistance gene is expressed only in females and confers resistance to puromycin.

In some embodiments, a genetically engineered male insect population is provided. It comprises a population of insects. In some embodiments, the population can comprise any of the insects provided herein. In some embodiments, the insects comprise a vector that encodes a protective protein. The functionality of the protective protein is dependent upon splicing of a sex-specific intron. Splicing of the sex-specific intron is determined by the male (or female) insect's sex determination machinery. The sex-specific intron is located within the gene encoding the protective protein. In some embodiments, the protective protein is encoded by an antibiotic resistance gene. In some embodiments, the male (or female) insect's sex determination machinery allows proper removal of the sex-specific intron during protein production such that the protective protein confers survival to the male (or female) insect in the presence of a selection event. In some embodiments, the selection event comprises administration of a selection molecule that is toxic and/or harmful to the male (or female) insect in the absence of the protective protein and/or lethal to the female (or male) insect in the absence of the protective protein. In some embodiments, the protective protein is a protein that confers resistance to an antibiotic and the selection event is a positive selection event. In some embodiments, the protective protein converts a toxic form of the selection molecule into a non-toxic form. In some embodiments, the protective constructs can be one or more of: 1) Mutant FabI gene (mFabI) from E. coli genome, which confers triclosan resistance to the host; 2) High expression Dihydrofolate Reductase (DHFR) confers resistance to Methotrexate; 3) High expression of blasticidin resistance gene from Bacillus cereus (bsr), which codes for blasticidin-S deaminase, which confers resitance to blasticidin; 4) High expression of the Sh ble gene, first isolated from Streptoalloteichus hindustanus, which confers resitance to Zeocin; 5) High expression of the hygromycin resistance gene which confers resistance to Hygromycin B; 6) histidinol histidinol dehydrogenase; 7) pyrimidine synthesis inhibitor PALA Cytosine deaminase; 8) Ouabain rat al isoform of Na1,K1-ATPase; and 9) Nourseothricin Nourseothricin N-Acetyl Transferase.

In some embodiments, insects containing such genes allow for a positive selection event to be employed instead of an ongoing negative selection event. Thus, in some embodiments, methods of creating or using such insects or insect populations do not include negative selection or ongoing negative selection (that is, survival of the population depends upon the presence of a molecule, and selection only occurs upon removal of the molecule).

As such individuals and populations can be useful in sterile form to prevent native populations from reproducing as effectively, the insect(s) can be sterile or sterilized. In some embodiments, the insect (or entire population) can be or has been irradiated to produce sterile male(s). In some embodiments, an entire population can be exposed to the selection event to select only a subpopulation of male(s), and then the entire subpopulation of males can be irradiated to produce a genetically engineered sterile population. In some embodiments, the subpopulation can be chemically sterilized.

In some embodiments, the genetically engineered insects can be produced by transforming a starting insect population with one or more transformation vectors, such as plasmids. For example, the target insects can be transformed with a vector that includes targeting sequences that enable the vector to integrate into the genome of the insect and gene encoding an antibiotic resistance protein for positive selection.

In some embodiments, the protective protein gene (such as an antibiotic gene) has a sex-specific intron that is excised in a sex-specific manner during protein production and generates a functional protective protein in a sex specific manner. The functional protein confers resistance to a selection agent or event in either males or females.

The methods and components described herein can be applied to various types of insects. For example, and without limitation, the insect can be a direct pest or indirect pest. As used herein, “direct pests” refers to insects that can cause damage at one or more stage of their life cycle by, for example, eating crops or damaging animals. The New World screw-worm fly Cochliomyia hominivorax, for example, is a direct pest of cattle, and the spotted wing Drosophila, Drosophila suzukii is pest of many fruit crops. As used herein, “indirect pests” refers to insects that transmit human diseases, for example, mosquitoes which carry malaria. Indirect pests of organisms other than humans, such as livestock or plants are also known.

Additional examples of insects include, but are not limited to, Asian citrus psyllid (diaphorini citriii, Australian sheep blowfly (Lucilia cuprina, Asian tiger mosquito (Aedes albopictus); Japanese beetle (Popilla japonica), White-fringed beetle (Graphognatus spp.), Citrus blackfly (Aleurocanthus woglumi), Oriental fruit fly (Dacus dorsalis), Olive fruit fly (Dacus oleae), tropical fruit fly (Dacus cucurbitae, Dacus zonatus), Mediterranean fruit fly (Ceratitis capitata), Natal fruit fly (Ceratitis rosa), Chemy fruit fly (Rhagoletis cerasi), Queensland fruit fly (Bactrocera tryoni), Caribbean fruit fly (Anastrepha suspensa), imported fire ants (Solenopis richteri, Solenopis invictai, Gypsy moth (Lyman tria dispar), Codling moth (Cydia pomonella), Brown tail moth (Euproctis chrysorrhoea), yellow fever mosquito (Aedes aegypti), malaria mosquitoes (Anopheles gambiae, Anopheles stephansi), New world screwworm (Cochliomyia hominivorax), Old World Screwworm (Chrysomya bezziana), Tsetse fly (Glossina spp), Boll weevil (Anthonomous grandis), Damsel fly (Enallagma hageni), Dragonfly (Libellula luctuosa), and rice stem borer (Tryporyza incertulas). In some embodiments, the insect either transmits human disease or are agricultural pests. In some embodiments, the insects are wild insect populations. In some embodiments, the insects are mosquitoes or flies (for example fruit flies). The mosquitoes can be, for example, Aedes sp. or Anopheles sp. In some embodiments, the mosquito is yellow fever mosquito (Aedes aegypti), malaria mosquito (Anopheles gambiae, Anopheles stephensi), and Asian tiger mosquito (Aedes albopictus). In some embodiments, the insect is one that transmits a disease of a mammal. The disease can be any disease, for example, malaria and/or yellow fever. In some embodiments, the insect is a Spotted wing Drosophila (Drosophila Suzukii).

In some embodiments, a method of using a first insect population for control, suppression or elimination of a second insect population is provided. In some embodiments, the method comprises providing a first insect population that comprises a genetically engineered sterile male insect population. This can be any of the insects or insect populations provided herein. In some embodiments, the genetically engineered sterile insect population is generated from a starting insect population. The starting insect population can be genetically engineered with a vector comprising components that allow for a short-term positive sex-specific selection and generation of a male insect population. In some embodiments, the male insect population is further exposed to radiation to generate a genetically engineered sterile male insect population that is used in a desired area. The desired area contains a second insect population that is a wild insect population. In some embodiments, this suppresses the second insect population in the desired area thus achieving a desired level of control.

In some embodiments, the second population of insects is one that transmits human disease or is an agricultural pest. In some embodiments, the insect population is any of those provided herein.

In some embodiments, the insect population to be controlled, suppressed, or eliminated is the same as the starting insect population. In some embodiments, the insect population to be controlled, suppressed, or eliminated is a species that is different but related to and can be controlled, suppressed, or eliminated by the starting insect population species.

In some embodiments, the second insect population is eventually reduced by a desired amount. In some embodiments, the desired amount can be 1, 5, 10, 20, 30, 40, 50, 60, 70, 80, 90, 95, 98, 99, or 100%, including any range above any one of the preceding values and any range between any two of the preceding values. In some embodiments, this can occur over the time course of days, weeks, months, or years. In some embodiments, this can occur over generations of the second population, for example, 1, 2, 3, 4, 5, 6, 7, 8, 9, 10 or more generations can result in the desired decrease. In some embodiments, additional additions of the first population can be added to the area to be treated. In some embodiments, multiple rounds of application of the first insect population is performed, for example, 2, 3, 4, 5, 6, 7, 8, 9, 10, 20, 30, 40, 50 or more rounds of administration can be performed.

In some embodiments, a method of using a first insect population for control of a second insect population is provided. The method comprises providing a first insect population that comprises a gene encoding a protective protein. The gene comprises a non-native intron within the gene, and the first insect population comprises a genetically engineered sterile male insect population. The method further comprises applying the first insect population to a desired area. The desired area contains a second insect population. This allows for control, suppression or elimination of the second insect population in the desired area.

In some embodiments, the desired area refers to a geographical area where the first insect population is to be released to control, suppress or eliminate the second insect population. The desired area can comprise, for example, a household, park, a crop field, wetlands, a town, an urban area. The desired level of control can be about 55%, about 60%, about 65%, about 70%, about 75%, about 80%, about 85%, about 90%, about 91%, about 92%, about 93%, about 94%, about 95%, about 96%, about 97%, about 98%, about 99% or 100%.

In some embodiments, the approaches described herein allow for the scalable production of homozygous transgenic insects expressing two engineered sex-specific antibiotic resistance genes (or any gene encoding a set of protection proteins).

In some embodiments, this can be puromycin N-acetyltransferase gene (pac) and neomycin 3′-phosphotransferase (neo). The pac gene confers resistance to puromycin. Puromycin is an aminonucleoside antibiotic produced by the bacterium Streptomyces alboniger. It inhibits protein synthesis by disrupting peptide transfer on ribosomes causing premature chain termination during translation. It is a potent translational inhibitor in both prokaryotic and eukaryotic cells. The neo gene confers resistance to neomycin and geneticin (G418). G418 antibiotic is an aminoglycoside commonly used as a selective agent for eukaryotic cells as it interferes with the function of 80S ribosomes and protein synthesis. Both of these antibiotic resistance genes have been shown to confer antibiotic resistance in Drosophila (Iwaki et al., 2003; Steller and Pirrotta, 1985).

To utilize these antibiotic resistance genes for insect sex selection, they (or any other protective protein) can be engineered to encode functional proteins whose function is dependent upon the proper splicing of known sex specific introns that are contingent on the insect's sex determination machinery. To ensure this dependence, these introns can be strategically placed within the protein-coding frames of the antibiotic resistance genes. This results in the production of a functional protein only when the intron is correctly spliced out of the mRNA transcript. For the scenarios in which the intron fails to be correctly spliced out, stop codons inhibit translation and a truncated non-functional protein is produced.

In this configuration, if the introns are not spliced at all, or correctly, a functional protein is not produced and antibiotic resistance is not conferred. The sex specific introns used can be any that will serve the functions as outlined herein.

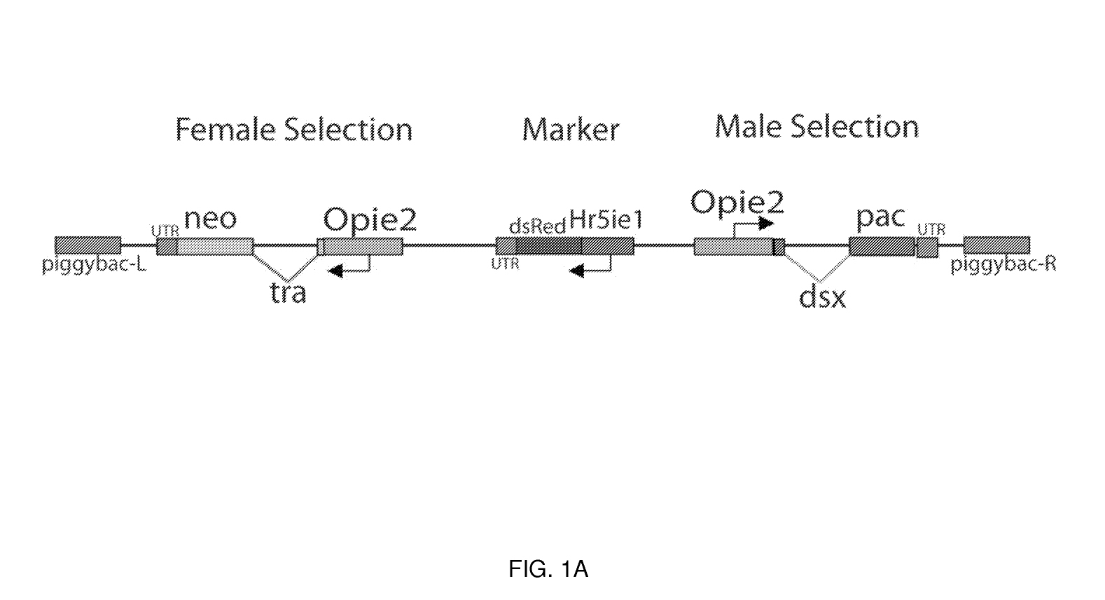

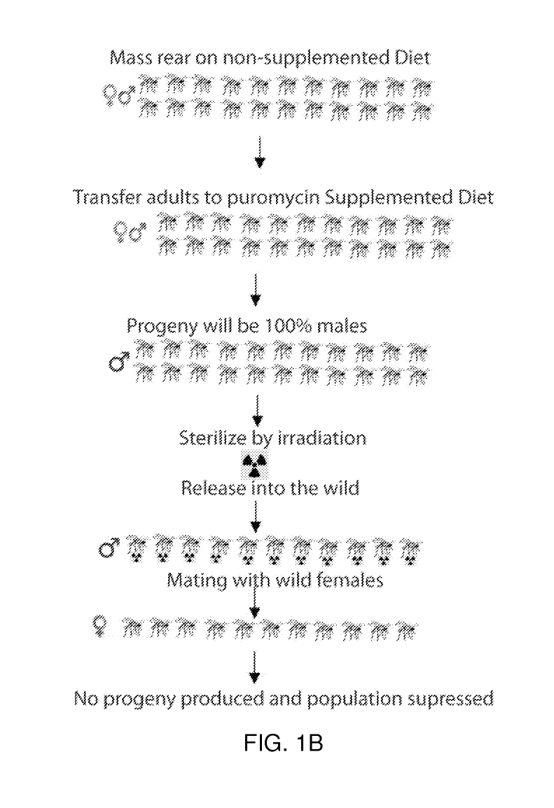

In some embodiments, one can use drosophila dsx and transformer (tra) that were previously characterized in Drosophila (Pomiankowski et al., 2004). The dsx male specific intron can be incorporated into the puromycin resistance gene, and the female specific tra intron can be incorporated into the G418 resistance gene (FIG. 1a). These engineered sex-specific antibiotic resistance (or protective protein) genes can be separately expressed, from a single piggyback transposable element (TE), throughout the insect from two separate copies of Opie2 regulatory sequences that originate from the baculovirus Orgyia pseudotsugata multicapsid nuclear polyhedrosis virus (OpMNPV) (Theilmann and Stewart, 1992) (FIG. 4a).

In some embodiments, one or more of the disclosed embodiments employs the piggybac TE element. This has been shown to be portable across many species of insects (Handler, 2002).

In some embodiments, one or more of the disclosed embodiments employs puromycin, G418, which is effective in all eukaryotic cells, indicating that it will likely work in most, if not all, insects. Additionally, the resistance genes for these antibiotics are clearly established, are effective, and should also be portable across a wide variety of insects.

In some embodiments, one or more of the disclosed embodiments employs the Opie2 and Hr5ie1 regulatory sequences that promote expression of the sex-specific antibiotic resistance genes and the transformation marker, respectively, originate from a baculovirus known to infect a large variety of insects. In some embodiments, any promoter that expresses at high levels ubiquitously can be employed. For example, in some embodiments actin or ubiquitin can be employed as a promoter.

In some embodiments, one or more of the disclosed embodiments employs drug selection that occurs at the first instar stage, allowing for gender to be sorted early. After gender sorting antibiotics, surviving individuals can then transferred to normal food. This saves money on drug costs as first instar larvae consume very little, and also limits the period of exposure to the drug.

In some embodiments, one or more of the disclosed embodiments employs insects that are reared on antibiotic selection that are resistant to the toxic effects of the antibiotic and are therefore healthy and have high fitness compared to insects that are not so reared.

In some embodiments, one or more of the disclosed embodiments is inherently resistant to breakage resulting from mutation, translocation and recombination. For example, if the antibiotic resistance gene mutates and is nonfunctional, this mutation will be lost when the host harboring this mutation is exposed to the antibiotic as these hosts will not survive and therefore this mutation would be selected against. Also, if the female-specific antibiotic resistance cassette (Opie2-dsxneo) recombines to the Y-chromosome, this cassette will be non-functional in males as males lack the machinery to properly splice the dsx intron, resulting in these individuals not surviving when exposed to the antibiotic.

In some embodiments, one or more of the disclosed embodiments employs an underlying mechanism for how the sex-specific antibiotic (or, more generally, protective protein) expression is conserved across many species of insects. In some embodiments, the male specific sex splicing of the dsx intron is highly conserved across many insects, indicating it will be straightforward to transfer this positive male selection system into many other insects (Pomiankowski et al., 2004).

In some embodiments, unlike mechanical separation which is ineffective for some species of mosquitoes including the malaria vectors (Anopeheles genus), which lack significant discriminating pupal size differences, the present embodiments can be employed. Similarly, a limitation with mechanical size separation approaches for insects is that these are entirely species specific and will not work in most insects and require optimal rearing conditions. In contrast, the various embodiments presented herein can avoid such issues.

An alternative classical approach for efficient genetic sex separation (GSS) that has been effectively used for Anopheles mosquitoes is to link a conditionally lethal allele to the Y chromosome through irradiation-induced chromosomal rearrangements (Curtis et al., 1976). For example, resistance to dieldrin (Rdl), an insecticide that blocks γ-aminobutyric acid receptors inhibiting transport of chloride ions, has been translocated to the Y chromosome in multiple Anopheles species allowing for the permissive survival of only males when exposed to dieldrin (Baker et al., 1981; Curtis, 1978; Curtis et al., 1976; Lines and Curtis, 1985; Robinson and Pham, 1987). Despite the fact that these lines have a high ˜1% recombination frequency (i.e. loss of dieldrin selection for males) and must therefore be maintained with additional selection, they have been shown to be scalable. For example, surviving dieldrin resistant Anopeheles arabiensis males can be semi-sterilized using irradiation and potentially used in SIT type approaches (Yamada et al., 2012). In addition to Y-Linked dieldrin resistance in mosquitoes, there are more examples of insecticides used for a similar purpose including Y-Linked propoxur and malathion resistance in other mosquito species (Kim et al., 1987; Mcdonald and Asman, 1982; Seawright et al., 1978; Shetty, 1987). Another example of an effective translocation based GSS strain that does not use insecticides for sorting, is a temperature sensitive lethal (tsl) allele linked to the Y chromosome in the medfly (Kerremans and Franz, 1995). This approach relies on exposing the eggs to increased temperatures, when gender sorting is desired, resulting in the female eggs selectively dying (Kerremans and Franz, 1995). While these translocation-based approaches can be effective, they require a considerable amount of effort and good fortune to produce. Translocations also induce large fitness costs on the organism as large chromosomes have been rearranged and these fitness costs reduce mating competitiveness. Additionally, they are unstable and can easily break down if the selection allele translocates or recombines away from the Y chromosome. Therefore, they all require additional selection and maintenance, and if the translocation/recombination frequency is high this can be detrimental to this approach. Finally, these approaches are also not portable across species and must be produced one species at a time. Various embodiments provided herein can address one or more of these issues.

Another GSS approach has relied on generating transgenic insects that express sex-linked fluorescent markers that can be mechanically sorted. This approach has been implemented in the Mediterranean fruit fly by generating transgenic strains harboring selectable markers linked to the Y chromosome (Condon et al., 2007). In addition to sex-linked systems, sex-limited expression systems have also been developed. For example, in A. Gambaie a sex-specific doublesex (dsx) intron was encoded in eGFP resulting in eGFP specific expression exclusive to males (Magnusson et al., 2011). Sex separation was achieved mechanically by using an optical fluorescent COPAS sorting system (Marois et al., 2012). While fluorescent sex sorting does work, in general all fluorescence-based sex-sorting approaches suffer the disadvantage that each larva needs to be individually examined and sorted. While instruments for automatic mechanical separation have been developed, they are generally not suitable for large-scale SIT programs and will require further development to reduce costs and increase throughput. Various embodiments provided herein can address one or more of these issues.

Alternatively, sex separation could be achieved by negative selection against females using sex-specific repressible lethal traits, whereby altering rearing conditions could result in the elimination of females. As noted above, a recently developed modification to SIT technology known as Release of Insects carrying a Dominant Lethal (RIDL) has been developed by the British based-forprofit-company Oxitec (Thomas et al., 2000). In this system recombinant DNA technology is used to introduce into insects a gene cassette that confers dominant, drug-repressible, lethality. Oxitec has developed this technology into two separate systems: 1) in bisex RIDL all progeny with at least one copy of the transgene die in absence of the drug. This version requires a downstream sex sorting mechanism. 2) Female specific RIDL-only female progeny with at least one copy of the transgene die in absence of the drug (they are negatively selected). These systems have been produced in multiple insect species including crop pests such as Pink bollworm moth (Pectinophora gossypiella), Diamondback moth (Plutella xylostella), olive fruit fly (Bactrocera oleae), medfly (Ceratitis capitata), Mexican fruit fly (Anastrepha ludens), and in disease vectors such as the Dengue Mosquito (Aedes aegypti), and the Asian tiger mosquito (Aedes albopictus) (Ant et al., 2012; Fu et al., 2010; Gong et al., 2005; Labbe et al., 2012; Morrison et al., 2012). In proof-of principal experiments it has been demonstrated that one can successfully reduce wild populations (Harris et al., 2012). While female specific RIDL is an effective negative-selection approach for genetic sexing it relies on the progeny being provided a diet supplemented with large amounts of tetracycline. Tetracycline has numerous unwanted side effects on insects, such as loss of the gut microbiome, loss of symbiotic bacteria, and effects on mitochondrial function. Thus, not surprisingly, the feeding of tetracycline has recently been shown to impose a large fitness cost in insects (Zeh et al., 2012). Various embodiments provided herein can address one or more of these issues.

In summary, in each of the many examples of various sex separation technologies that have been tried, each technology and approach has its own limitation. Various embodiments provided herein provide for a technology that is easily portable across various species of insects that incorporates a universal, scalable, sex-separation technology, that does not induce a large fitness cost to the organism or rely on mechanical separation devices. The following examples, including the experiments conducted and results achieved, are provided for illustrative purposes only and are not to be construed as limiting the present invention.

### Example 1

To make a genetically engineered insect, a transformation vector was constructed with targeting elements that allowed the transformation vector to integrate into the genome. In the version of the vector in FIG. 1A, the targeting elements were piggyBac inverted terminal repeats that flanked a central region. The central region of the transformation vector contained two antibiotic resistance genes neo and pac that confer resistance to neomycin and puromycin, respectively. The antibiotic resistance genes also contained sex-specific introns within their coding frame. The neo gene contained the tra intron and the pac gene contained the dsx intron. Given the sex-specific splicing of tra and dsx introns (tra in females and dsx in males), the neo gene and pac gene would only be expressed in females and males, respectively. Thus, only the females would be resistant to neomycin and only the males would be resistant to puromycin. The central region also contained a transformation marker (in this example, dsRed which encodes a fluorescent protein) that allowed for the detection and separation of genetically engineered insects. The baculovirus promoter Opie2 was used for the neo and pac genes and the Hr5ie1 promoter was used for the dsRed gene. This transformation vector was introduced into the germline of the fruit fly Drosophila.

### Example 2

In order to make a genetically engineered sterile male insect population, transformation of the germline of the insects with the transformation vector from Example 1 was followed by expansion of the insects on a diet that was not supplemented with a selection molecule (for example, an antibiotic). The expanded insect population containing both males and females was transferred to a diet supplemented with puromycin. Following reproduction, the female progeny were selectively killed at the first instar larval stage of development owing to the inability to express the puromycin resistance gene thus resulting in only the genetically engineered males surviving to adulthood. The male fraction obtained following antibiotic selection was sterilized by irradiation.

### Example 3

Introduction of the genetically engineered sterile male insect population of Example 2 in the wild will result in the mating of the sterile males with the female populations in the wild. Because no progeny will be produced, there will be control and suppression of the population. Continually releasing sterile males in the wild will thus result in the eventual elimination of the insect population in the wild.

The method of this example is depicted as a flowchart in FIG. 1B.

### Example 4

As proof of principle, an embodiment of this system was developed for the fruit fly Drosophila melanogaster. As an initial test of antibiotic toxicity, Drosophila wild-type larvae were separately fed increasing amounts of either G418 or puromycin. Both antibiotics were highly toxic around 1 mg/ml such that when larvae were fed antibiotic-supplemented diet at this concentration, 100% first instar death was observed (n=10,000). Thus, these two antibiotics can be used as selection molecules in this system.

### Example 5

To determine if the toxicity of puromycin and neomycin could be negated, antibiotic resistance genes pac and neo were used. Two types of transgenic flies were generated each expressing one type of antibiotic resistance gene under the control of the promoter Opie2. One type of transgenic fly expressed the pac gene and the other expressed the neo gene. Transgenic flies were fed a diet supplemented with increasing concentrations of either puromycin or G418, respectively. The antibiotic resistance genes were able to dominantly negate the toxicity of the respective antibiotics and allowed the transgenic flies to survive. Additionally, these transgenic flies were quite healthy with high fitness. Thus, the pac and neo genes could be used to counter the toxicity of G418 and neomycin, respectively.

### Example 6

The possibility of utilizing the endogenous sex determination machinery to promote sex-specific antibiotic resistance-based selection was next tested. Sex-specific introns dsx and tra were incorporated into the coding frames of the antibiotic resistance genes pac and neo, respectively, and two types of flies were generated, one type expressing Opie2-tra-neo and another type expressing Opie2-dsx-pac. The dsx intron can be excised by splicing only in males and the tra intron can be excised by splicing only in females. This precise sex-specific splicing of these two introns is dictated by the sex determination machinery of the fly. The placement of these introns resulted in a functional pac protein only produced in males (Opie2-dsx-pac), and a functional neo protein only produced in females (Opie2-tra-neo), allowing for sex selection upon exposure to either puromycin or G418, respectively. The Opie2-tra-neo expressing flies were fed diet containing increasing concentrations of G418 resulting in 100% female progeny at ˜5 mg/ml (n=2,000). The Opie2-dsx-pac expressing flies were fed diet containing increasing concentrations of puromycin resulting in 100% male progeny at ˜2 mg/ml (n=500).

Thus, the sex-specific introns tra and dsx can be used to bring about a sex specific expression of the antibiotic resistance genes pac and neo.

### Example 7

The sex-specific selection of Example 6 was shown to be splicing-dependent but drug independent by developing a sex specific selection for female flies expressing Opie2-tra-pac. When larvae were fed diet containing increasing concentrations of puromycin, the progeny obtained were 100% females (n=500). Thus, the sex specific introns can be interchangeably used with either antibiotic resistance genes to select for either males or females.

### Example 8

Several working models based on this system have been developed. To collect females, the Opie2-tra-neo and Opie2-tra-pac systems were developed. With these two systems, one can select for 100% females using G418 or puromycin supplemented diet, respectively.

To collect males, the Opie2-dsx-pac system was developed, which allows us to select 100% males using puromycin supplemented diet.

A version in which the two selection systems (Opie2-tra-neo and Opie2-dsx-pac) have been combined into a single transformation vector such that the genetically engineered insects would contain both antibiotic resistance markers (FIG. 1A) has also been developed.

However, in this version, the G418 resistance gene would only be expressed in the females and the puromycin resistance would only be expressed in the males.

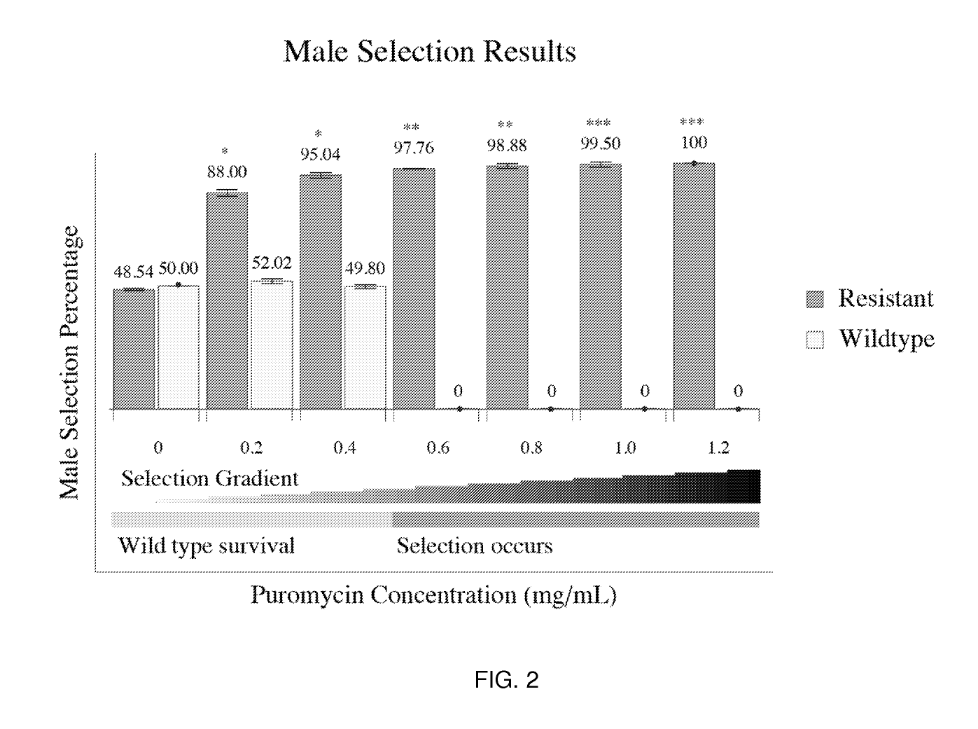

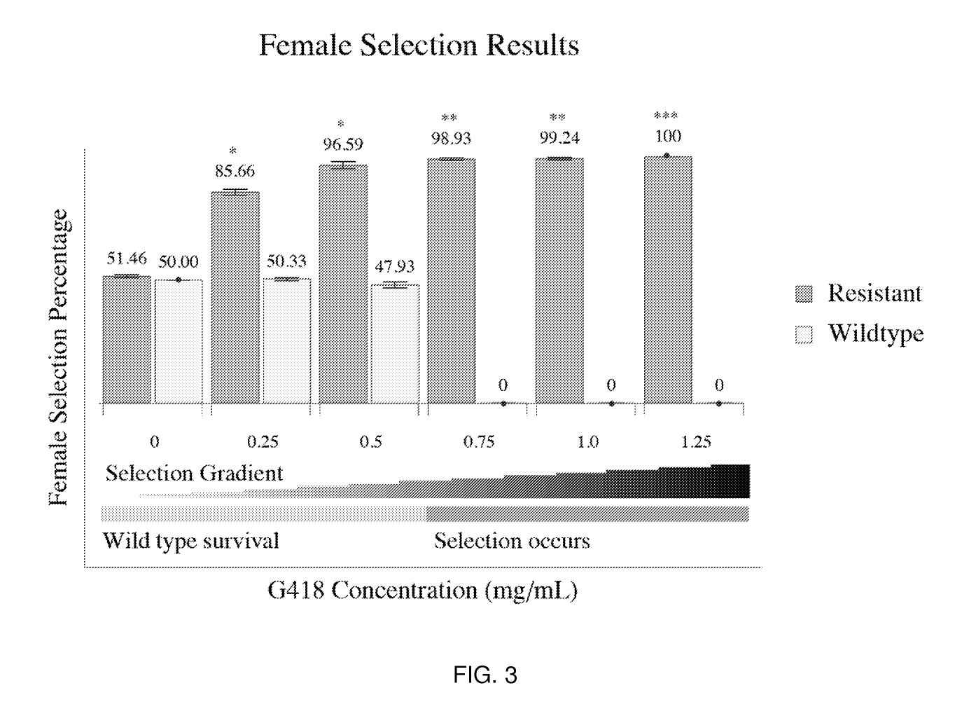

The specificity of expression was demonstrated by selecting 100% males on diet supplemented with increasing concentrations of puromycin (FIG. 2) and selecting 100% females on diet supplemented with increasing concentrations of G418 (FIG. 3). Overall, this is a fully synthetic positive drug sex-selection system engineered in insects, with the principals and components highly conserved, making this concept portable across a wide range of insect species.

While the above system has been developed using the antibiotic resistance genes pac and neo, the concept of using sex specific introns as a positive sex selection approach can work for a wide range of other types of drug or insecticide resistance genes that are no longer commonly used in the field. In some embodiments, these can include the cytochrome p450 genes that have been shown to confer resistance to a wide range of insecticides including DDT, nitenpyram and dicyclanil (Daborn et al., 2007).

### Example 9

As shown above, in the absence of an antibiotic supplemented diet, genetically engineered transgenic fit homozygous males and females can easily be mass reared. When male selection is desired for SIT, heterozygous and homozygous mixed sex adults can be transferred to puromycin-supplemented diet. The females will oviposit their eggs in this food. Diet supplemented with puromycin will selectively kill all female progeny at the first instar larval stage of development because of their inability to splice the male specific dsx intron and express a functional pac resistance gene, thus resulting in only males surviving to adulthood. After antibiotic sex sorting, adult males can then be sterilized by irradiation and continually released in the wild in high numbers. The sterile males would mate with wild females and suppress the insect populations in the wild (FIG. 1B).

### Example 10

In order to make the system outlined in Example 10 more universal and widely applicable, it is configured such that it can be performed using G418-supplemented diet to selectively kill all males at the first instar stage of development because of their inability to splice the female specific tra intron and express a functional neo resistance gene, thus resulting in only females surviving to adulthood.

### Example 11

This system can be applied to the Anopheles genus. Female mosquitoes of some of the species of the Anopheles genus transit malaria to humans. Given that females are the disease-spreading gender in the case of malaria, it would be more desirable to select the males using this method and release the males in a desired area to bring about control of the wild insect population.

Genetically engineered Anopheles mosquitoes can be generated such that they contain the pac gene with the dsx sex-specific intron. Adults of both sex can be transferred to puromycin-supplemented diet. Following reproduction to a desired population level, the puromycin-supplemented diet will selectively kill all female progeny at the first instar larval stage of development because of their inability to splice the male specific dsx intron and express a functional pac resistance gene. This results in only males surviving to adulthood.

### Example 12

Genetically engineered Anopheles mosquitoes can be generated such that they contain the neo gene with the dsx sex-specific intron. Adults of both sex can be transferred to G418-supplemented diet. Following reproduction, the G418-supplemented diet will selectively kill all female progeny at the first instar larval stage of development because of their inability to splice the male specific dsx intron and express a functional neo resistance gene, thus resulting in only males surviving to adulthood. After. antibiotic (puromycin or G418)-based sex sorting, adult males can be sterilized by irradiation and continually released in the wild in high numbers.

The sterile males would mate with wild females and thus bring about suppression of wild insect populations.

The foregoing description and Examples detail certain specific embodiments of the invention and describes the best mode contemplated by the inventors. It will be appreciated, however, that no matter how detailed the foregoing may appear in text, the invention may be practiced in many ways and the invention should be construed in accordance with the appended claims and any equivalents thereof. While the present teachings have been described in terms of these exemplary embodiments, the skilled artisan will readily understand that numerous variations and modifications of these exemplary embodiments are possible without undue experimentation. All such variations and modifications are within the scope of the current teachings. The foregoing examples are provided to better illustrate the disclosed teachings and are not intended to limit the scope of the teachings presented herein. All references cited herein, including patents, patent applications, papers, text books, and the like, and the references cited therein, to the extent that they are not already, are hereby incorporated by reference in their entirety. In the event that one or more of the incorporated literature and similar materials differs from or contradicts this application, including but not limited to defined terms, term usage, described techniques, or the like, this application controls. The foregoing written specification is considered to be sufficient to enable one skilled in the art to practice the invention. The foregoing description and examples detail certain preferred embodiments of the invention and describe the best mode contemplated by the inventors. It will be appreciated, however, that no matter how detailed the foregoing may appear in text, the invention may be practiced in many ways and the invention should be construed in accordance with the appended claims and any equivalents thereof.

1. A method of making a genetically engineered insect population, the method comprising:
transforming a starting insect population with a transformation vector to create a genetically engineered insect population;
expanding the genetically engineered insect population in the absence of any selection; and
applying a sex-specific selection event to select for a male fraction of the genetically engineered insect population.

2.-7. (canceled)

8. A method of using a first insect population for control of a second insect population, the method comprising:
providing a first insect population, wherein the first insect population comprises a gene encoding a protective protein, wherein the gene comprises a non-native intron within the gene, and the first insect population comprises a genetically engineered sterile male insect population; and
applying the first insect population to a desired area, wherein the desired area contains a second insect population, thereby controlling, suppressing or eliminating the second insect population in the desired area.

9.-13. (canceled)

14. A transformation vector comprising:
a targeting sequence that allows for insertion of the transformation vector in a genome wherein the genome comprises an insect genome;
an antibiotic resistance gene;
a sex-specific intron positioned within the antibiotic resistance gene, wherein the intron is excisable by splicing in a sex-specific manner, wherein the excision of the sex-specific intron allows the antibiotic gene to be expressed in a sex-specific manner; and
a regulatory element.

15. The transformation vector of claim 14, wherein the expression of the antibiotic resistance gene confers resistance to short-term positive sex-specific selection.

16. The transformation vector of claim 14, wherein the targeting sequence is an inverted terminal repeat.

17. The transformation vector of claim 14, wherein the targeting sequence is a piggyBac inverted terminal repeat.

18. The transformation vector of claim 14, further comprising a gene that encodes an enzyme that allows the insertion of the transformation vector in the genome.

19. The transformation vector of claim 18, wherein the enzyme is a recombinase.

20. The transformation vector of claim 18, wherein the enzyme is a transposase.

21. The transformation vector of claim 14, wherein the antibiotic resistance gene confers resistance to neomycin.

22. The transformation vector of claim 14, wherein the antibiotic resistance gene confers resistance to puromycin.

23. The transformation vector of claim 14, wherein the intron is from the tra gene.

24. The transformation vector of claim 14, wherein the intron is from the dsx gene.

25. The transformation vector of claim 14, wherein the antibiotic resistance gene comprises a tra intron and confers resistance to neomycin upon sex-specific splicing in females but not in males.

26. The transformation vector of claim 14, wherein the antibiotic resistance gene comprises a tra intron and confers resistance to puromycin upon sex-specific splicing in females but not in males.

27. The transformation vector of claim 14, wherein the antibiotic resistance gene comprises a dsx intron and confers resistance to neomycin upon sex-specific splicing in males but not in females.

28. The transformation vector of claim 14, wherein the antibiotic resistance gene comprises a dsx intron and confers resistance to puromycin upon sex-specific splicing in males but not in females.

29. The transformation vector of claim 14, wherein the transformation vector further comprises a transformation marker to identify a genetically engineered insect.

30. The transformation vector of claim 14, wherein the regulatory element comprises a promoter and a terminator.

31. A genetically engineered insect comprising a sex-specific selection element, the sex-specific selection element comprising:
an antibiotic resistance gene comprising a sex-specific intron, wherein the intron is excisable by splicing in a sex-specific manner, wherein excision of the sex-specific intron allows the antibiotic resistance gene to be expressed in a sex-specific manner, and wherein expression of the antibiotic resistance gene confers resistance to a positive sex-specific selection.

32.-40. (canceled)

41. A genetically engineered insect comprising:
a nonnative gene that encodes for a protective protein, wherein the normative gene comprises an intron that is not native to the gene encoding the protective protein, wherein the intron is excisable by splicing in a sex-specific manner, wherein excision of the intron allows the protective protein to be expressed in a sex-specific manner, and wherein expression of the protective protein confers resistance to a selection event.
